SEQUENCE LISTING (21 sequences)

SEQ ID NO: 1 - protein, 413 aa, Mus musculus
  MISC_FEATURE (1)..(413): amino acid sequence for NORE1 protein
MASPAIGQRPYPLLLDPEPPRYLQSLGGTEPPPPARPRRCIPTALIPAAGASEDRGGRRSGRRDPEPTPRDCRHARPVRPGLQPRLRLRPGSHRPRDVRSIFEQPQDPRVLAERGEGHRFVELALRGGPGWCDLCGREVLRQALRCANCKFTCHSECRSLIQLDCRQKGGPALDRRSPGSTLTPTLNQNVCKAVEETQHPPTIQEIKQKIDSYNSREKHCLGMKLSEDGTYTGFIKVHLKLRRPVTVPAGSGPSPSMDAIKEVNPAATTDKRTSFYLPLDAIKQLHISSTTTVSEVIQGLLKKFMVVDNPQKFALFKRIHKDGQVLFQKLSIADYPLYLRLLAGPDTDVLSFVLKENETGEVEWDAFSIPELQNFLTILEKEEQDKIHQLQKKYNKFRQKLEEALRESQGKPG

SEQ ID NO: 2 - DNA, 3018 bp, Mus musculus
  misc_feature (31)..(1269): nucleotide sequence encoding NORE1 protein
gtagctgcgccgctgactgaggccttggccatggcttccccggccatcgggcaacgtccctacccgctgctcctagatcccgagccgccgcggtatctgcagagtctgggtggcaccgagccgccacctcccgcccggccgcgccgctgcatccccacggccctgatccccgcggccggggcgtcagaggatcgcggtggcaggaggagtggccggagggaccccgaacccacgccccgagactgccgacacgctcgccctgtccggcccggtctgcagccgagactgcggctgcgacctgggtcacaccgaccccgcgacgtgaggagcatcttcgagcagccgcaggatccccgcgtcttggccgagagaggcgaggggcaccgtttcgtggaactggcgctgcggggcggtccgggctggtgtgacctgtgcggacgagaggtgctgcggcaggcgctgcgctgcgctaattgtaaattcacctgccactcggagtgccgcagcctgatccagttggactgcagacagaaggggggccctgccctggatagacgctctccaggaagcaccctcaccccaaccttgaaccagaatgtctgtaaggcagtggaggagacacagcacccgcccacgatacaggagatcaagcagaagattgacagctataacagcagggagaagcactgcctgggcatgaagctgagtgaagatggcacctacacaggtttcatcaaagtgcatttgaagctccgacggccagtgacggtgcccgctggatccggccccagtccatctatggatgccattaaggaagtgaaccctgcagccaccacagacaagcggacttccttctacctgccactcgatgccatcaagcagctacatatcagcagcaccaccacggttagtgaggtcatccaggggctgctcaagaagttcatggttgtggacaacccacagaagtttgcactttttaagcggatacacaaagatggacaagtgctcttccagaaactctccattgctgactatcctctctaccttcgtctgctcgctgggcctgacaccgatgttctcagctttgtgctaaaggagaatgaaactggagaggtggagtgggatgccttttccattcctgaactccagaactttttaactatcctggaaaaagaggagcaggacaagatccatcaactgcaaaagaagtacaacaaattccgtcagaaactggaagaggcattacgagagtcccaagggaagccggggtaaccagccgacttcctgtcctctcagtgccctccaatttattttattgttaattattttgcaacaaagagttactgttaagacacctctggtggttccaccagtcgcctgcccagcagttaacagatgtggcacaaagtctcttccacgcagtgtctatgcagggttccgattcctgctaacccaccacaccatggctctggagagcttcccgcctgggatcagaactcctgtggaatgaccagtgtttccctgctcagtctgctggcctctcagaaaccaaatagttgcctctctggtcaccaaactccaatcaatcaccagccggcaaaaggaaagaaaggtttcagagcctgtgtgttctttctctggatttactcttcagttcctcttttggtttgtttggttggttttttttggccacgtatagtatatttaaggatcaaatgtggcatattcattctagctaagtccttgaaagcaggaaaatgctcatgaaaggactgtccttgccccaaggtgcctcttcttctctagtactagacactcagggtcagcctgagatttcaagaggctacagcctgaccaggccgtcttcttattacccagcaggctgtgtgcatgcaaacccaaagacatatatgcacatctgtgtggtatttcagcatgtctctgtccaatgtttgatatgttaacatttgaatttaatgctgtcctccttatgggtttctaccaaagagaaaccagccacttatcaattttagtttcttgctgagctgccagaaagtattacagagaagcacatccaagctgtctgtggcctacgcctgcagggggtggggggcctgaatctccttggccttcagttccacctccacctctggctttagggtctccagctgttgcctgagtagtagctttgattacagcggtaaagtcctccaacttggagtcctttctggtgggaagcatggtctgctcgcagcacagcactgagcagacccgtgggcctgacttccctggtgacttcagtgcctttttgtttgcagagaaaagagtggggcactttgcttgaagctctctgctggcttgcccctggcaggaagtggacaatggtgctatagagccaaggacacagcctcagagcacagggtgattgatgatcagcctctttcccatcaagcttcccggtcaggctttgactttgaagatgcgaggttactagactgcattgacagcatcagattatgactccaactcttgagtagttcagacttaaaaccaatcagccagagtagccaggactgcaaagacactcaatacagatggagaaaaacttgtccctttaaaagagggccagtgtttcaattgagcctccagaggagaccactttcatgttgtgcttgcctttccataccctttcctcgggttgttttaagcccaagcttctccgtgtagcctaaaaagttccctaccagcccagctgaagccacactgctcccgtcccagaagaacgccaaatccttgtcattcaaactgtgcatcgtttgcagagctgcaaaaagcaacatgagctagcgactctgaggttgtgcacgccatcagccccttggctgcctgaggtctcatgcccagccttacacctctctcccttaagaagcccccgtcctgctgtgtactacaggggcacgtggaatcattcccttcatcctgcatgtctgtagcgttaggagaaggcatggctcctgc

SEQ ID NO: 3 - protein, 340 aa, Homo sapiens
  MISC_FEATURE (1)..(340): amino acid sequence for RASSF1A protein
MSGEPELIELRELAPAGRAGKGRTRLERANALRIARGTACNPTRQLVPGRGHRFQPAGPATHTWCDLCGDFIWGVVRKGLQCAHCKFTCHYRCRALVCLDCCGPRDLGWEPAVERDTNVDEPVEWETPDLSQAEIEQKIKEYNAQINSNLFMSLNKDGSYTGFIKVQLKLVRPVSVPSSKKPPSLQDARRGPGRGTSVRRRTSFYLPKDAVKHLHVLSRTRAREVIEALLRKFLVVDDPRKFALFERAERHGQVYLRKLLDDEQPLRLRLLAGPSDKALSFVLKENDSGEVNWDAFSMPELHNFLRILQREEEEHLRQILQKYSYCRQKIQEALHACPLG

SEQ ID NO: 4 - DNA, 1020 bp, Homo sapiens
  misc_feature (1)..(1020): nucleotide sequence encoding RASSF1A protein
atgtcgggggagcctgagctcattgagctgcgggagctggcacccgctgggcgcgctgggaagggccgcacccggctggagcgtgccaacgcgctgcgcatcgcgcggggcaccgcgtgcaaccccacacggcagctggtccctggccgtggccaccgcttccagcccgcggggcccgccacgcacacgtggtgcgacctctgtggcgacttcatctggggcgtcgtgcgcaaaggcctgcagtgcgcgcattgcaagttcacctgccactaccgctgccgcgcgctcgtctgcctggactgttgcgggccccgggacctgggctgggaacccgcggtggagcgggacacgaacgtggacgagcctgtggagtgggagacacctgacctttctcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttggg

SEQ ID NO: 5 - protein, 270 aa, Homo sapiens
  MISC_FEATURE (1)..(270): amino acid sequence for RASSF1C protein
MGEAEAPSFEMTWSSTTSSGYCSQEDSDSELEQYFTARTSLARRPRRDQDEPVEWETPDLSQAEIEQKIKEYNAQINSNLFMSLNKDGSYTGFIKVQLKLVRPVSVPSSKKPPSLQDARRGPGRGTSVRRRTSFYLPKDAVKHLHVLSRTRAREVIEALLRKFLVVDDPRKFALFERAERHGQVYLRKLLDDEQPLRLRLLAGPSDKALSFVLKENDSGEVNWDAFSMPELHNFLRILQREEEEHLRQILQKYSYCRQKIQEALHACPLG

SEQ ID NO: 6 - DNA, 810 bp, Homo sapiens
  misc_feature (1)..(810): nucleotide sequence encoding RASSF1C protein
atgggcgaggcggaggcgccttctttcgaaatgacctggagcagcacgacgagcagtggctactgcagccaagaggactcggactcggagctcgagcagtacttcaccgcgcgaacctcgctagctcgcaggccgcgccgggaccaggacgagcctgtggagtgggagacacctgacctttctcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttggg

SEQ ID NO: 7 - protein, 615 aa, Caenorhabditis elegans
  MISC_FEATURE (1)..(615): amino acid sequence for T24F1.3 protein
MLRSVVNNNDKAKESEQSVRVATDDPTYQSYAFQPSTSSSNISWFGGLRTKLSQLSLFPDWMSQLGIKSDDEKMETSSTSSPQSEQSIGEYDESTLVFRPVQLDHFGGAGQEDDGEQVDIWDFGATFLADLDSNHVWNDTIERQHFGPLKDLITTGGGANKINNHSFKTHSLLHPTWCDKCGDFIWGILKEALKCEHCNYTCHARCRDLVTLDCRSPGSSLASSTEFDSIYPQLDGTLGTIPKGLILPPAMSSSTGSDKENGNGNSAGISAENPIFSVKNSFTLPKSFSPVDSLRTKEPSAPPESRYATLRVVERYVKEDTPFEWTDEYKEMDLERKIHSYNSLAKGMEITLHEDGVNFGGHIHVNMNLSRPISVVQGVIPPTVYDVVNTAKSTAKTTSLRTITSFFLPRNTAKVINIDSKTTARKMIVTLLKKFRVADNPRKFALYECEQITDEATCTLNRKLTRISDDACPLKVVLNWQSPHCGRALVLQENDTGDILWDAFEIPELENFLRILGMEEKQYVFQTQQKYQQYRYHLDAELRQRGHSVPDAAAQPMVQTNPFLDEEFLRNQDEYGTSDSMLFSGTIKNAIMGEDPDYVNLEYLKKQNMDQSTNL

SEQ ID NO: 8 - DNA, 1845 bp, Caenorhabditis elegans
  misc_feature (1)..(1845): nucleotide sequence encoding T24F1.3 protein
atgcttcggtccgttgtaaataataatgataaagctaaagagagtgaacaaagtgtccgtgtggcgactgacgacccaacctaccaatcatacgccttccaaccgtcgacatcttcatctaacattagctggttcggaggtcttcgaacaaaactctcacaattgagtctgtttcccgattggatgtcacagttgggaataaaatcggacgacgagaaaatggagacatcatcgacaagtagcccacaaagtgagcaatcaattggcgagtacgatgagagtacactcgtctttaggccagtacagcttgatcattttggtggagcagggcaggaggatgatggagagcaggttgatatctgggatttcggcgccacatttctcgccgatttggattcgaaccacgtttggaatgatacaatagaacggcaacactttggccctctgaaagacctgataacaactggaggaggagcaaataagataaataatcacagtttcaaaacacattccctactacatccaacatggtgcgacaagtgcggagatttcatttggggtattctgaaggaagcgctcaagtgtgaacattgtaactacacctgccatgcaagatgtcgtgacttagtgacccttgactgtagatctcctggaagctctctcgcttcttcaactgaatttgactcaatttatccacaacttgacggtactctcggaaccattccaaaaggtcttattcttccaccagcaatgagctctagcactggatctgataaggaaaacggcaatggaaattcagcagggatttcagcagagaatccaattttttccgtgaaaaattcctttacattacccaaatccttttcaccagttgatagtctcaggacgaaagaaccaagtgctccaccagagtccagatatgccacgttaagagttgtggaaagatatgtaaaggaagatacaccatttgagtggactgatgagtacaaggaaatggatctcgaacggaaaatccattcctataactctttggcaaagggaatggaaatcacattacatgaggacggtgtcaactttggaggtcacattcatgtcaatatgaatttgagccgaccgattagtgttgttcaaggagtcattccaccaactgtttatgatgttgtgaatactgcaaagtcgacggcaaaaaccacatctttaagaacaattacttcatttttcttaccgagaaacactgcaaaagtgataaatatcgatagcaagacaactgcgagaaaaatgattgtaacactgctgaagaagttccgagttgccgataacccgaggaagtttgcactttatgagtgcgaacagattactgatgaggctacatgtacattgaacagaaagctaacgagaatatctgacgacgcgtgtcctctaaaagttgtgctcaattggcagagcccacattgtggccgagctttggtgttgcaggaaaacgacactggtgacattctgtgggacgcttttgaaattcccgaattggaaaacttcttgcgaattttgggaatggaggagaagcagtacgtcttccaaactcaacagaaatatcaacaatacaggtatcatcttgatgcggaactccgtcaacgaggtcattcggttcccgatgcagccgcgcagccgatggttcagaccaatccatttttagatgaagagttccttcgcaaccaggatgagtatggaacatcagacagtatgctcttctcgggaacaatcaagaacgctatcatgggagaagacccggactatgtgaatctcgagtatctcaagaaacaaaatatggaccagtcaacgaatttg

SEQ ID NO: 9 - protein, 487 aa, Mus musculus
  MISC_FEATURE (1)..(487): amino acid sequence for MST1 protein
METVQLRNPPRRQLKKLDEDSLTKQPEEVFDVLEKLGEGSYGSVYKAIHKETGQIVAIKQVPVESDLQEIIKEISIMQQCDSPHVVKYYGSYFKNTDLWIVMEYCGAGSVSDIIRLRNKTLTEDEIATILQSTLKGLEYLHFMRKIHRDIKAGNILLNTEGHAKLADFGVAGQLTDTMAKRNTVIGTPFWMAPEVIQEIGYNCVADIWSLGITAIEMAEGKPPYADIHPMRAIFMIPTNPPPTFRKPELWSDNFMDFVKQCLVKSPEQRATATQLLQHPFVKSAKGVSILRDLINEAMDVKLKRQEAQQREVDQDDEENSEEDEMDSGTMVRAAGDEMGTVRVASTMSGGANTMIEHGDTLPSQLGTMVINTEDEEEEGTMKRRDETMQPAKPSFLEYFEQKEKENQINSFGKNVSGSLKNSSDWKIPQDGDYEFLKSWTVEDLQKRLLALDPMMEQEMEEIRQKYRSKRQPILDAIEAKKRRQQNF

SEQ ID NO: 10 - DNA, 1461 bp, Mus musculus
  misc_feature (384)..(384): n = a,c,g or t
atggagaccgtgcagctgaggaacccaccgcgcaggcagctgaaaaagttggatgaagatagtttaaccaagcagcccgaggaagtgtttgatgtcttagagaagcttggagaggggtcttatggcagcgtgtacaaggctattcataaagagactggccagattgttgcaatcaagcaagtgcccgtggaatcggacctgcaggagataatcaaggaaatctccatcatgcagcagtgtgacagccctcacgtagtcaagtattatggcagctactttaagaacacagacctgtggattgttatggagtactgtggggctggttctgtatctgatatcattcggctacggaacaagacgttaacagaagatgaaatagctacnatattacaatcaactcttaagggactagaataccttcattttatgagaaaaatacaccgagatatcaaggcgggaaatattttgctaaacacagaaggccatgcaaagcttgcagactttggagtcgcaggtcaacttacagataccatggcaaagaggaatacagtaatagggaccccgttttggatggctcctgaagttattcaggaaattgggtacaactgtgtggcagacatctggtctctgggaataactgccatagaaatggctgaaggaaagcccccatatgctgatatccatccaatgagggcaatattcatgattcctacaaaccctcctcccacattccgtaagccagagctgtggtcagacaacttcatggattttgtgaagcagtgtctggtaaaaagcccggagcagagagccacagccactcagctcctacagcacccgtttgttaagagtgccaaaggagtgtcaatattgcgagacttaattaacgaagccatggatgtgaaactgaagcgccaggaagcccagcagcgggaagtggaccaggacgacgaggagaactcagaggaggatgaaatggattccggcacgatggttcgagctgccggtgatgagatgggcactgtccgagtagccagcacgatgagcggaggagccaatactatgattgagcacggtgacacgttgccgtcccagctgggcaccatggtgatcaacacggaggacgaggaagaggaaggaaccatgaaaagaagagatgagaccatgcagcctgcaaaaccgtctttccttgaatactttgaacaaaaagaaaaggaaaatcagatcaacagctttggcaagaatgtatctggctctctgaagaattcttccgattggaaaatccctcaggatggagactatgagtttctcaagagctggactgtggaggaccttcagaagaggctcttggccctcgaccccatgatggaacaggaaatggaagagatccggcagaagtaccggtccaagcggcagccaatcctggatgccattgaagccaagaagaggaggcaacagaacttc

SEQ ID NO: 11 - protein, 10 aa, Homo sapiens
  MISC_FEATURE (1)..(10): amino acid sequence of a c-myc epitope tag peptide
EQKLISEEDL

SEQ ID NO: 12 - protein, 188 aa, Homo sapiens
  MISC_FEATURE (1)..(188): amino acid sequence of Ki-Ras(G12V) protein
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 13 - DNA, 564 bp, Homo sapiens
  misc_feature (1)..(564): nucleotide sequence encoding Ki-Ras(G12V) protein
atgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatg

SEQ ID NO: 14 - protein, 189 aa, Homo sapiens [sequence omitted]

SEQ ID NO: 15 - DNA, 567 bp, Homo sapiens
  misc_feature (1)..(567): nucleotide sequence of Ha-Ras(G12V) protein
atgacggaatataagctggtggtggtgggcgccgtcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcc

SEQ ID NO: 16 - protein, 12 aa, Artificial Sequence
  amino acid sequence for a FLAG epitope tag peptide
MDYKDDDDKNSA

SEQ ID NO: 17 - protein, 10 aa, influenza virus
  MISC_FEATURE (1)..(10): amino acid sequence for a hemagglutinin (HA) epitope tag peptide
MYPYDVPDYA

SEQ ID NO: 18 - protein, 16 aa, Artificial Sequence
  amino acid sequence for a c-Src myristoylation and polybasic peptide
MGSSKSKPKDPSQRRR

SEQ ID NO: 19 - protein, 18 aa, Homo sapiens
  MISC_FEATURE (1)..(18): amino acid sequence for a membrane association (CAAX) peptide of Ki-Ras4B
SKDGKKKKKKSKTKCVIM

SEQ ID NO: 20 - protein, 648 aa, Homo sapiens [sequence omitted]

SEQ ID NO: 21 - DNA, 1944 bp, Homo sapiens
  misc_feature (1)..(1944): nucleotide sequence encoding c-Raf1 protein
atggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttc

(19) United States
(12) Patent Application Publication
(10) Pub. No.: US 2005/0064384 A1
(43) Pub. Date: Mar. 24, 2005

(54) Recruitment of mst1 or mst2 protein kinase to induce apoptosis in eukaryotic cells

(76) Inventors: Joseph Avruch, Brookline, MA (US); Xian-Feng Zhang, Somerville, MA (US); Andrei Khokhlatchev, Charlottesville, VA (US)

Correspondence Address: Leon R Yankwich;Yankwich & Associates, 201 Broadway, Cambridge, MA, 02139, US

(21) Appl. No.: 10/498,698

(22) PCT Filed: Dec. 16, 2002
(86) PCT No.: PCT/US02/40102

Related U.S. Application Data
(60) Provisional application No. 60/340,418, filed on Dec. 14, 2001.

(51) Int. Cl. C12Q 1/00; C12Q 1/68

(52) U.S. Cl. 435/4; 435/6

(57) ABSTRACT

Methods of identifying or evaluating a test compound for the ability to induce an apoptotic pathway involving recruitment of protein kinase MST1 or MST2 in eukaryotic cells are described.

### STATEMENT AS TO U.S. GOVERNMENT SPONSORED RESEARCH

The invention described herein was supported in whole or in part by National Institutes of Health grant GM51281. The United States Government has certain rights in the invention.

### GENERAL FIELD OF THE INVENTION

This invention is generally in the field of apoptosis. In particular, this invention relates to compositions and methods for modulating a Ras-mediated apoptotic pathway in eukaryotic cells.

### BACKGROUND

Cells of all multicellular, eukaryotic organisms, from the simple nematode Caehnorhabditis elegans, to vertebrates, including humans, appear to possess one or more similar or analogous molecular signaling pathways that regulate a mechanism for cell death. Such programmed cell death, known as apoptosis, is essential for proper development of tissues and organs, e.g., by deleting certain tissue structures at proper times in development, by ablating entire fields of cells, as in sculpting features and organs, and by regulating the number of cells in a system, such as in the nervous system. In normal, healthy cells, a variety of positive and negative control molecules, i.e., cell trophic factors, regulators, adapters, and effectors, interact to control apoptosis. A loss or decrease in the relative amounts of cell trophic factors and/or negative (suppressor) apoptosis control molecules is typically sufficient for apoptosis to commence and result in cell death. However, unlike cell death due to necrosis or cell lysis, apoptotic cell death is an orderly process characterized by condensation of chromosomes, shrinkage in cell size, nuclear fragmentation, blebbing of the membrane and cell fragmentation into apoptotic cell fragments, which are readily phagocytized by other cells and without lysis and release of intracellular contents that could harm surrounding cells and tissues or induce inflammation.

Studies have revealed that in vertebrates, such as mammals, apoptosis occurs in a series of steps of a pathway leading to the activation of one or more cysteine proteases, known as caspases, which degrade intracellular structures leading to cell death. Current models of apoptosis in vertebrates feature a variety of protein molecules that interact or affect one another to ultimately regulate caspases. Such proteins involved in apoptosis include Bad, Bcl-2, Bcl-x1, 14-3-3, Akt, Apaf1 and Bax (see, e.g., section 23.8 “Cell Death and Its Regulation”, In Molecular Cell Biology, Lodish et al., eds., (W. H. Freeman and Co., New York, 2000), pp. 1044-1050).

Ras was originally identified in a mutant form as an oncogenic protein and has recently also been found to regulate apoptosis. Ras is a small GTPase (guanosine triphosphate cleavage enzyme), which is embedded on the inner (cytoplasmic) surface of the eukaryotic cell plasma membrane and whose primary function is to relay proliferative and developmental signals downstream of cell surface receptors, especially, but not exclusively, receptor tyrosine kinases (Barbacid, Ann. Rev. Biochem., 56: 851-891 (1987); Satoh et al., J. Biol. Chem., 267: 24149-24152 (1992) Shields et al., Trends Cell Biol., 4: 147-154 (2000)). Ras signaling is activated on binding GTP, which results in the reconfiguration of two epitopes on the cytoplasmic face of the Ras polypeptide thereby creating binding sites for Ras “effector” proteins (Marshall, Trends Biochem. Sci., 18: 250-254 (1993)). The first Ras effectors to be identified were the kinases of the Raf subfamily, which are activated consequent to binding Ras-GTP in vivo, and in turn activate the classic MAPK pathway (Avruch et al., Trends Biochem. Sci., 19: 279-283 (1994); Avruch et al., Recent Prog. Horm. Res., 56: 127-155 (2001)). It is now well recognized that Ras utilizes effectors in addition to the Raf kinases. Among the earliest evidence was the observation that although active mutants of both Ras and Raf can transform cell lines of fibroblastic origin (Barbacid, Ann. Rev. Biochem., 56: 851-891 (1987)), spontaneously occurring active Ras mutants are mostly found in human tumors of epithelial origin (Bos, Cancer Res., 49: 4682-4689 (1989)).

Whereas active Ras alone can transform epithelial cell lines, active Raf mutants do not (Oldham et al., Proc. Natl. Acad Sci. USA, 93: 6924-6928 (1996)). Certain Ras effector loop mutants that have lost the ability to bind and activate Raf and to transform fibroblasts nevertheless can collaborate with each other or with weakly active, nontransforming Raf mutants, to achieve cellular transformation (White et al., Cell, 80: 533-541 (1995); Joneson et al., Science, 271: 810-812 (1996)). With respect to cellular, wild type Ras, it is known that although at least two peaks of increased Ras GTP charging occur during G1 progression of the cell cycle, only the earlier peak is accompanied by an increase in MAPK activity; the relevant effector in mid-late G1 is not known (Taylor et al., Curr. Biol., 6: 1621-1627 (1996); Foschi et al., EMBO J., 16: 6439-6451 (1997)). Direct support for the existence of multiple Ras effectors comes from the identification of proteins, other than the Raf kinases, that can bind to Ras in a GTP-dependent manner, including, e.g., the protein kinases MEKK1 (Russell et al., J. Biol. Chem., 270: 11757-11760 (1995)) and PKCζ (Diaz-Meco et al., J. Biol. Chem., 269: 31706-31710 (1994)). Whereas the role of these protein kinases in Ras action is unknown, considerable evidence indicates that the catalytic subunits of the Type 1 PI-3 kinases are bona fide Ras effectors (Rodriquez-Viciana et al., Nature, 370: 527-532 (1994); Rodriquez-Viciana et al., Cell, 89: 457467 (1997)). These p110 polypeptides each bind specifically to Ras-GTP through a structurally conserved domain; mutation within this domain that interrupts Ras-GTP binding increases basal PI-3 kinase activity and significantly reduces RTK activation of PI-3 kinase activity in vivo (Rodriquez-Viciana et al., Nature, 370: 527-532 (1994), Rodriquez-Viciana et al., EMBO J., 15: 2441-2451 (1996)). A third class of probable Ras effectors is the family of guanylnucleotide exchange factors active on the Ral A GTPase (Ral-GDS, Rgl, Rlf). These polypeptides also bind selectively to Ras-GTP through a conserved noncatalytic domain (Wolthus, Curr. Opin. Genet. Dev., 1: 112-117 (1999)). In addition, several noncatalytic polypeptides have been isolated by their ability to bind specifically to Ras-GTP, including AF-6 (Kuriyama et al., J. Biol. Chem., 271: 607-610 (1996)), Rin1 (Han et al., Mol. Cell. Biol., 15: 1318-1323 (1995)), and NORE (Vavvas et al., J. Biol. Chem., 273. 5439-5442 (1998)).

As noted above, Ras was first identified in mutant, active form as a retroviral-transforming agent. Moreover, mutations conferring constitutive Ras activation are found in nearly 30% of all human tumors (Bos, Cancer Res., 49: 4682-4689 (1989)). Although active Ras mutants are able to transform nearly all immortalized cell lines, they are usually unable to transform normal primary cells (Newbold et al., Nature, 304: 648-651 (1983)), except in the presence of a cooperating oncogene (Hirakawa et al., Proc. Natl. Acad. Sci. USA, 85: 1519-1523 (1988); Ridley et al., EMBO J., 7: 1635-1645 (1988); Lui et al., Cell, 70: 153-161 (1992)) or in association with the loss of certain tumor suppressor genes (Lloyd et al., Curr. Opin. Genet. Dev., 8: 4348 (1998); Serrano et al., Cell, 88: 593-602 (1997)). Introduction of constitutively active Ras into primary cells generally results in cell cycle arrest mediated by increased levels of a variety of cyclin-dependent kinase inhibitors, or in apoptosis (Serrano et al., Cell, 88: 593-602 (1997); Downward, Curr. Biol., 8: 49-54 (1998); Frame et al., Curr. Opin. Genet. Dev., 10:106-113 (2000)). Downregulation of the pathways mediating cell cycle arrest and/or apoptosis is probably crucial to the expression of Ras-induced oncogenesis. The mechanism by which Ras promotes G1 cell cycle arrest has received considerable attention, whereas the mechanism by which constitutively active Ras promotes apoptosis was incompletely understood. One mechanism appears to involve increased levels of the p53 tumor suppressor protein. Oncogenic Ras strongly stimulates the activation of p53 through the induction of p14^{ARF}, which neutralizes the p53 inhibitor, MDM2 (Palmero et al., Nature, 395: 125-126 (1998); Bates et al., Nature, 395: 124-125 (1998)). The ability of active alleles of Ras to cause apoptosis of primary mouse embryo fibroblasts (MEF) is greatly diminished by homozygous deletion of the gene encoding p53 (Fukasawa et al., Mol. Cell. Biol., 17: 506-518 (1997)). The existence of additional, p53-independent pathways for Ras-induced apoptosis is indicated by the ability of RasG12V to promote apoptosis if the Ras-induced increase in NFκb activity is suppressed (Mayo et al., Science, 278: 1812-1815 (1997); Joneson et al., Mol. Cell. Biol., 19: 5892-5901 (1999)) either in p53^{-/-} mouse embryo fibroblasts, or in 53^{+/+} NIH3T3 cells expressing p53^{v135}, a temperature-sensitive dominant inhibitor of p53. Phorbol ester-induced downregulation of PKCs induces apoptosis in Ras transformed cells (Liou et al., J. Biol. Chem., 275: 39001-39011 (2000)). Thus, as is true for Ras-induced proliferation, multiple pathways exist for Ras induction of apoptosis would appear to be present in the eukaryotic cell.

Clearly, the identification of specific apoptotic pathways in eukaryotic cells, of critical components of such pathways, and of the specific interactions among such components, would offer the potential for identifying new compounds that act through a defined mechanism to destroy cells that are proliferating at an undesirable rate and location, such as occurs in cancer.

### SUMMARY

The present invention provides methods to identify compounds that induce a Ras-mediated apoptotic pathway in eukayotic cells. The invention is based on the discovery of a pathway by which Ras initiates apoptosis through the direct recruitment of the proapoptotic protein kinase, MST1 or MST2. The effector protein NORE can bind Ras and MST1 (or MST2) to form a stable complex in vivo. NORE molecules are able to associate with one another to form homodimers or with RASSF1 molecules, which also bind MST1 (or MST2) but not Ras, to form heterodimers. Such homodimeric or heterodimeric complexes are able to localize MST1 (or MST2) to the plasma membrane and thereby activate an apoptotic pathway leading to cell death. In fact, according to the invention, an increased expression of any of the effector proteins described herein may increase the level of complex formation to effectively translocate MST1 (or MST2) to the cell membrane to induce apoptosis, particularly in cells that normally express relatively low levels of one or more of the effector proteins. With the elucidation of the interaction and involvement of critical effector proteins, such as NORE, MST1, MST2, and RASSF1A, in forming stable complexes in vivo that induce a Ras-mediated apoptotic pathway provides the basis for methods of identifying or evaluating new compounds that can specifically induce this apoptotic pathway. Such new compounds find particular use in killing cells that are undergoing an undesirable rate of growth, such as in cancer. Accordingly, new compounds identified or evaluated by the methods of the invention are particularly attractive as anti-cancer drugs that act by inducing a specific apoptotic pathway in cancer cells.

In one embodiment, the invention provides a method of identifying or evaluating a compound that induces a Ras-mediated pathway of apoptosis in eukaryotic cells comprising:
a) providing eukaryotic cells having the genetic information for expressing a NORE protein, an MST1 or MST2 protein kinase, a Ras protein, and, optionally, an RASSF1A protein;
b) contacting said eukaryotic cells with a test compound; and
c) assaying said eukaryotic cells contacted with said test compound for an increased level of formation of a complex comprising NORE, MST1 or MST2, and Ras proteins or, optionally, of RASSF1A, MST1 or MST2, and Ras proteins, compared to the level of formation of said complex in cells not contacted with said test compound;
   wherein an increase in the level of said complex formation indicates that said test compound induces a Ras-mediated apoptotic pathway in eukaryotic cells.

In another embodiment, the invention provides a method of identifying or evaluating a compound that induces a Ras-mediated pathway of apoptosis in eukaryotic cells comprising:
a) providing eukaryotic cells having the genetic information for expressing a NORE protein;
b) contacting said eukaryotic cells with a test compound; and
c) assaying said eukaryotic cells contacted with said test compound for increased expression of NORE protein or increased synthesis of NORE-encoding mRNA compared to cells not contacted with said compound;
   wherein an increase in NORE protein expression or in synthesis of NORE-encoding mRNA indicates that said test compound induces a Ras-mediated pathway of apoptosis.

In yet another embodiment, the invention provides a method of identifying or evaluating a test compound that induces a Ras-mediated pathway of apoptosis in eukaryotic cells comprising:
a) providing eukaryotic cells having the genetic information for expressing an RASSF1A protein;
b) contacting said eukaryotic cells with a test compound; and
c) assaying said eukaryotic cells contacted with said test compound for increased expression of RASSF1A protein or increased synthesis of RASSF1A-encoding mRNA compared to cells not contacted with said test compound;
   wherein an increase in RASSF1A protein expression or in synthesis of RASSF1A-encoding mRNA indicates that said test compound stimulates a Ras-mediated pathway of apoptosis in eukaryotic cells.

In yet another embodiment, the invention provides a method of identifying or evaluating a compound that stimulates a pathway of apoptosis in eukaryotic cells comprising:
a) providing eukaryotic cells having the genetic information for expressing an MST1 or MST2 protein kinase;
b) contacting said eukaryotic cells with a test compound;
c) assaying said eukaryotic cells contacted with said test compound for increased expression of MST1 or MST2 protein or increased synthesis of MST1 or MST2-encoding mRNA compared to cells not contacted with said compound;
   wherein an increase in MST1 or MST2 expression or in synthesis of MST1 or MST2-encoding mRNA indicates that said test compound stimulates a pathway of apoptosis in eukaryotic cells.

Methods of the invention for identifying or evaluating a test compound for the ability to induce a Ras-mediated apoptotic pathway may comprise providing a eukaryotic cell that expresses a fusion protein, wherein the fusion protein comprises a portion of the amino acid sequence of a particular effector protein linked in frame to an epitope tag sequence (e.g., HA tag, c-myc tag, FLAG tag, and the like) for which high affinity antibodies are readily available and, thereby, provide a particularly sensitive and convenient assay for detecting the ability of a test compound to alter effector protein expression or complex formation.

In a preferred embodiment, the eukaryotic cells employed in the methods of the invention are Ras-transformed eukaryotic cells or other mammalian cells.

A test compound so identified by a method described herein may be further tested for its ability to induce apoptosis in one or more cells of interest, such as one or more types of cancer cells.

The methods of the invention identify or evaluate compounds that may be applied to any of a variety of cells to inhibit unwanted cell proliferation in an animal, such as in a mammal, including a human. Examples of cells of unwanted proliferation include but are not limited to cancer cells and cells in a recognized pre-cancerous state. Particularly preferred are methods of the invention employed to identify or evaluate compounds having the ability to induce apoptosis in one or more types of cancer (tumor) cells, including but not limited to, lung cancer cells, melanoma cancer cells, myeloma cancer cells, leukemia cancer cells, teratoma cancer cells, kidney cancer cells, brain cancer cells, bone cancer cells, bladder cancer cells, epithelial cancer cells, breast cancer cells, colon cancer cells, testicular cancer cells, and prostate cancer cells.

### BRIEF DESCRIPTION OF THE DRAWINGS

FIGS. 1A-1D show results demonstrating that NORE and its Homologs RASSF1 and T24F1.3 bind MST1. FIG. 1A shows binding FLAG-NORE1 homologs by GST-MST1. COS-7 cells were transfected with either a vector (pEBG) encoding GST or a GST-MST1 fusion protein, together with FLAG-tagged NORE1, NORE (1-267) (i.e., amino acids 1-267 of SEQ ID NO:1), NORE (251-413) (i.e., amino acids 251-413 of SEQ ID NO:1), or the NORE homologs RASSF-1A (full length, i.e., amino acids 1-340 of SEQ ID NO:3) RASSF1A (1-119) (i.e., amino acids 1-119 of SEQ ID NO:3), RASSF1C, or a fragment of the C. elegans polypeptide T24F1.3 (247-601) (i.e., amino acids 247-601 of SEQ ID NO:7). GST and the various GST fusion proteins were purified using GSH-agarose and analyzed by immunoblotting after SDS-PAGE. The diagram (cartoon) above the panels of immunoblots indicates the domain features of NORE-related polypeptides and relative location of a 300 amino acid long segment of homology between the polypeptides. Upper immunoblot panel: proteins retained on GSH beads were probed with anti-FLAG (M2 monoclonal, Sigma, St. Louis, Mo.) antibody. Second panel: cell extracts probed with anti-FLAG antibody. Third and fourth panels: cell polypeptides and identifies the 300 amino acid segment of homology.

FIG. 1B shows results indicating MST1 binds NORE carboxy terminal to the NORE-RA domain. HEK293 cells were transfected with expression vectors encoding GST, GST-NORE (7-413) (i.e., containing amino acids 7-413 of SEQ ID NO:1), or GST-NORE (358-413) (i.e., containing amino acids 358-413 of SEQ ID NO:1), the latter at increasing levels as compared with GST-NORE (7-413), together with FLAG-tagged wild type (wt) MST1, or MST1 Leu444Pro(L444P) or MST1(L444P), with a frameshift after amino acid 449 that replaces the carboxy-terminal 38 amino acids of the MST1 with an unrelated 8 amino acid segment (L444PΔCT). GST and GST fusion proteins were isolated from cell extracts by adsorption to GSH-agarose and analyzed by immunoblotting after SDS-PAGE. Upper panel: proteins retained on GSH beads were probed with anti-FLAG antibody. Middle panel: cell extracts probed with anti-GST antibody (α-GST). Lower panel: cell extracts probed with anti-FLAG antibody (α-FLAG).

FIG. 1C shows that the MST1 carboxy terminal 32 amino acids are sufficient to bind FLAG-NORE1. COS-7 cells were transfected with expression vectors encoding GST or GST-MST1 (456-487) (i.e., containing amino acids 456-487 of SEQ ID NO:9) together with FLAG-NORE1. GST and GST-MST1 (456-487) were isolated on GSH-agarose and were analyzed by immunoblotting after SDS PAGE. Upper panel: proteins retained on GSH-agarose beads probed with anti-FLAG antibody. Middle panel: cell extracts probed with anti-GST antibody. Bottom panel: cell extracts probed with anti-FLAG antibody.

FIG. 1D shows NORE1 binds MST1, but not related kinases. HEK293 cell were transfected with expression vectors encoding HA-NORE 1 and FLAG-tagged MST1, GCK, or SOK as indicated. FLAG-tagged proteins were immunopreciptated with anti-FLAG M2-agarose, eluted with the excess of the FLAG peptide. The eluates were analyzed by immunoblotting after SDS-PAGE. Upper panel: proteins immunoprecipitated (IP) by FLAG-agarose probed with anti-HA antibody 12CA5 (α-HA). Middle panel: cell extracts probed with anti-FLAG antibody (α-FLAG). Lower panel: cell extracts probed with anti-HA antibody (α-HA).

FIGS. 2A and 2B show results indicating that a NORE-MST1 complex exists in vivo. FIG. 2A shows that endogenous NORE1 and MST1 form a constitutive complex in KB cells. KB cells were deprived of serum for 24 hours; fetal calf was added to 10%, and cells were sampled thereafter at the times indicated. Aliquots of the cell extracts were incubated with normal goat IgG or goat anti-MST1 IgG (α-MST1) as indicated. After harvest on protein A/G beads, extensive washing, and SDS-PAGE, the recovered proteins (IP) were subjected to immunobloting with a rabbit polyclonal anti-NORE1 antibody (α-NORE1) described in (Vavvas et al., J. Biol. Chem., 273: 5439-5442 (1998)). Right lane, KB cell extract.

FIG. 2B shows that endogenous MST1 binds recombinant HA-NORE1 and is displaced by excess recombinant MST1. COS-7 cells were transfected with vectors encoding a constant amount of HA-NORE1 and FLAG vector or increasing amounts of FLAG-MST1. The HA-NORE was recovered from cell extracts by immunoprecipitation with anti-HA antibody 12CA5 (α-HA) and was subjected to immunobloting for MST1 and FLAG as indicated. Top panel: proteins recovered in the α-HA immunoprecititates (IP) probed with anti-MST1 antibody (α-MST1). Second panel: proteins recovered in the α-HA immunoprecipitates probed with anti-FLAG antibody (α-FLAG). Third panel: cell extracts probed with anti-FLAG antibody (α-FLAG); note the increasing expression of FLAG-MST1. Lowest panel: cell extracts probed with anti-HA antibody (α-HA).

FIGS. 3A-3D show results demonstrating that active Ras recruits the NORE-MST1 complex. FIG. 3A shows that RasG12V binds MST1 through NORE1. COS-7 cells were transfected with vectors encoding FLAG-tagged versions of MST1 (FLAG-MST1), NORE1 (FLAG-NORE1), and Ha-RasG12V (FLAG-V12RAS), using the amounts indicated; each transfection contained 15 μg DNA. Aliquots of the cell extracts were mixed with anti-Ras Y13-238 monoclonal antibody coupled to agarose beads (Santa Cruz). After extensive washing and SDS-PAGE, the polypeptides recovered by Y13-238 (upper panel) and aliquots of the cell extract (lower panel) were subjected to anti-FLAG immunobloting.

FIG. 3B shows relative binding of Ha-RasG12V and Ki-RasG12V to NORE1, RASSF1A, and Raf (1-257) (i.e., amino acids 1-257 of SEQ ID NO:20) in vitro. HEK293 cells were transfected with pEBG (encoding GST) or pEBG-NORE (7-413) or pEBG Raf (1-257) or pEBG RASSF1A. Aliquots from extracts of these cells containing comparable amounts of GST and each of the GST fusion proteins were adsobed to GSH-agarose. pCMV5 encoding either FLAG Ki-RasG12V (FLAG-V¹² H-Ras) or FLAG Ha-RasG12V (FLAG-V¹² Ki-Ras) were expressed separately in HEK293 cells. Aliquots of extracts from these cells (as shown in the center panel) were adsorbed to the immobilized GST proteins; the beads were washed extensively and eluted with SDS. The left panel shows an anti-FLAG (α-FLAG) immunoblot, and the right panel shows an anti-GST (α-GST) blot of this SDS eluate.

FIG. 3C shows that binding of NORE1 to Ha-RasG12V with a normal or mutant effector loop. HEK293 cells were tansfected with HA-tagged Ha-Ras mutants G12V (lane 1), G12V T35S (lane 2), G12V E37G (lane 3), or G12V Y40C (lane 4) together with GST-NORE1 (7-413). The latter was isolated from cell extracts on GSH-agarose, washed, and analyzed by immunoblotting after SDS-PAGE. Upper panel: proteins retained on GSH beads were probed with mouse anti-Ras antibody (AB-2, Calbiochem) (α-Ras). Middle panel: cell extracts probed with anti-Ras antibody (α-Ras). Lower panel: cell extracts probed with anti-GST antibody (α-GST).

FIG. 3D shows that serum induces the association of endogenous MST1 with endogenous cRas. KB cells were deprived of serum for 24 hours and then stimulated by the addition of fetal calf serum to 10%; cells were sampled before and at the indicated timnes after serum readdition. Aliquots of the cell extracts were mixed with anti-Ras Y13-238 antibody (α-Ras) coupled to agarose beads (Santa Cruz). The cell extracts and the washed beads were subjected to SDS-PAGE and analyzed by immunoblotting, as indicated. Upper panel, left four lanes: proteins (IP) recovered with α-Ras Y13-238 immunoblotted with anti-MST1 antibody (Zymed) (α-MST1); upper panel, right four lanes: cells extract immunoblotted with anti-MST1 antibody. Lower panel: Y13-238 immunoprecipitates (IP) immunoblotted with mouse anti-Ras antibody (AB-2, Calibiochem) (α-Ras).

FIGS. 4A-4D show that membrane recruitment amplifies the proapoptotic effect of MST1. FIG. 4A shows the effects of NORE and MST1 on annexin V surface binding (“staining”) in NIH3T3 and Jurkat cell. The number of observations for each condition is indicated. Where n≧3, the standard error (SE) is shown; where n=2, the error bars indicate the range of values. An asterisk (*) indicates that p<0.001 versus vector; a plus sign (+) indicates that p<0.01 versus vector; a multiplication sign (×) indicates that p<0.001 versus MST; a number sign (#) indicates that p<0.05 versus MST. The efficiency of NIH3T3 transfection was about 33%, as determined by counting GFP-positive cells transfected with a GFP reporter. The Jurkat data are from a single experiment; additional Jurkat experiments, analyzed second method, are shown in FIG. 4B.

FIG. 4B shows the effects of NORE and MST1 on the survival of Jurkat cells. Three series of experiments are shown; the number (n) of individual experiments is indicated. In the series shown as n=5, this number of observations applies only to the transfection of 8 μg cDNA for MST1 and NORECAAX (NORE with CAAX membrane association segment); the cell survival values indicated for MST1, 12-20 μg, and for NORECAAX, 10 and 15 μg, represent the average of two experiments, and no error bars are shown for these values. The comparison of MST1 (K59R) to vector was done twice; the error bar represents the range of values observed. Otherwise, standard errors (SE) are shown. An asterisk indicates that p<0.001 versus vector; a plus sign (+) indicates that p<0.01 versus vector; a multiplication sign (×) indicates that p<0.05 versus vector.

FIG. 4C shows the effects of NORE and MST1 on cell death in HEK293 cells. A set of three experiments is summarized. The standard error (SE) is shown. An asterisk indicates that p<0.001 versus vector; a multiplication sign indicates that p<0.05 versus vector.

FIG. 4D shows that the caspase inhibitor zVAD-FMK suppresses NORE1 and MST1-induced annexin V binding. NIH3T3 cells were transfected in duplicate with vectors encoding either NORECAAX together with wild type MST1, or with myrMST1. zVAD-FMK (42 μM) was added at the time of transfection, as indicated. The cells were harvested 24 hours later, stained with annexin V-Alexa 488, and analyzed by FACS as described in the Examples.

FIGS. 5A-5C show that Ki-RasG12V and Ha-RasG12V, E37G induced apoptosis through a NORE-MST1 Complex. FIG. 5A shows that Ki-RasG12V and Ha-RasG12V, E37G induce death in HEK923 cells, which is suppressed by interfering fragments (CT) of NORE and MST. The number of observations for each condition is indicated on the right. The SE is shown, except for n=2, where error bars indicate the range of values. An asterisk indicates that p<0.001 versus vector; a plus sign indicates that p<0.0001 versus Ki-RasG12V; a multiplication sign indicates that p<0.001 versus Ha-RasG12V, E37G.

FIG. 5B shows the effect of various caspase inhibitors on Ki-RasG12V induced death in HEK293 cells. Cells were transfected with empty vectors or vectors encoding Ki-RasG12V alone, or together with vectors encoding p35 CrmA catlytic inactive mutants of individual caspases: Caspase 3 (DNC3), Caspase 6 (DNC6), Caspase 7 (DNC 7), Caspase 8 (DNC 8), or Caspase 9 (DNC 9). The error bars represent the standard deviation (SD) of replicate determinations in a single experiment.

FIG. 5C shows that constitutively active variants of the Pi 3-kinase catalytic subunit suppress Ki-RasG12V-induced apoptosis. The values shown are the mean and standard deviation of triplicate measurements from a single experiment. A second experiment gave similar results.

FIG. 6 shows suppression of colony formation in non-small lung cancer cells transfected with the pcDNA3.1-NORE1 plasmid and expressing the encoded NORE1, compared to colony formation by control lung cancer cells transfected with the pcDNA3.1 vector alone.

See text for additional details of the experiments yielding the data depicted in the Figures.

### DETAILED DESCRIPTION

As noted above, the Ras protein is a small GTPase embedded on the cytoplasmic surface of the eukaryotic cell plasma membrane whose primary function is to relay proliferative and developmental signals downstream of cell surface receptors, especially, but not exclusively, receptor tyrosine kinases. Ras-mediated signaling is activated on binding GTP, which results in the reconfiguration of two epitopes on the cytoplasmic face of the Ras polypeptide thereby creating binding sites for Ras “effector” proteins, which specifically stimulate signaling down particular signaling pathways to direct the cell to carry out various Ras-mediated events (Marshall, Trends Biochem. Sci., 18: 250-254 (1993)). Such Ras-mediated events have recently been shown to include Ras-mediated apoptosis (programmed cell death) (Serrano et al., Cell, 88: 593-602 (1997); Downward, Curr. Biol., 8: 49-54 (1998), Guo et al., Curr. Opin. Cell Biol., 11: 745-752 (1999); Frame et al., Curr. Opin. Genet. Dev., 10: 106-113 (2000)).

As shown herein, this invention is based on the discovery of a new Ras-mediated pathway of apoptosis, including the identification of critical effector proteins of this new apoptotic pathway and various complexes of such effector proteins that form and induce this specific apoptotic pathway in eukaryotic cells. The invention uses this new information as the basis for new methods of identifying or evaluating compounds that specifically induce this new Ras-mediated pathway for apoptosis in various eukaryotic cells.

Proagoptotic NORE-Related Effectors of Ras-Mediated Apoptosis

As shown herein, formerly unknown protein complexes and functional equivalents thereof have been found comprising positive effector proteins, i.e., inducers, of a Ras-mediated apoptotic pathway in eukaryotic cells. Such effector protein complexes include a complex comprising a NORE protein or functional portion thereof that is non-covalently associated with an MST1 protein kinase and Ras. Such complexes are proapoptotic effector complexes, which recruit MST1 to the plasma membrane to induce a Ras-mediated apoptotic pathway in eukaryotic cells.

The effector protein NORE1 is a 413 amino acid noncatalytic polypeptide that contains several proline-rich motifs between amino acids 17 and 108, a central C1 zinc finger motif (amino acids 118-165 of SEQ ID NO:1), and a Ras association (RA) domain in its carboxy terminal segment (amino acids 267-358 of SEQ ID NO:1) (see, Vavvas et al., J. Biol. Chem., 273: 5439-5442 (1998)). Although it was previously shown that NORE binds directly to Ras-GTP and is recruited to cRas in vivo in response to EGF (Vavvas et al., J. Biol. Chem., 273: 5439-5442 (1998)), the biologic role and biochemical functions of NORE were not previously elucidated. An amino acid sequence for NORE1 is shown in SEQ ID NO:1, and a nucleotide sequence encoding NORE1 is shown in SEQ ID NO:2.

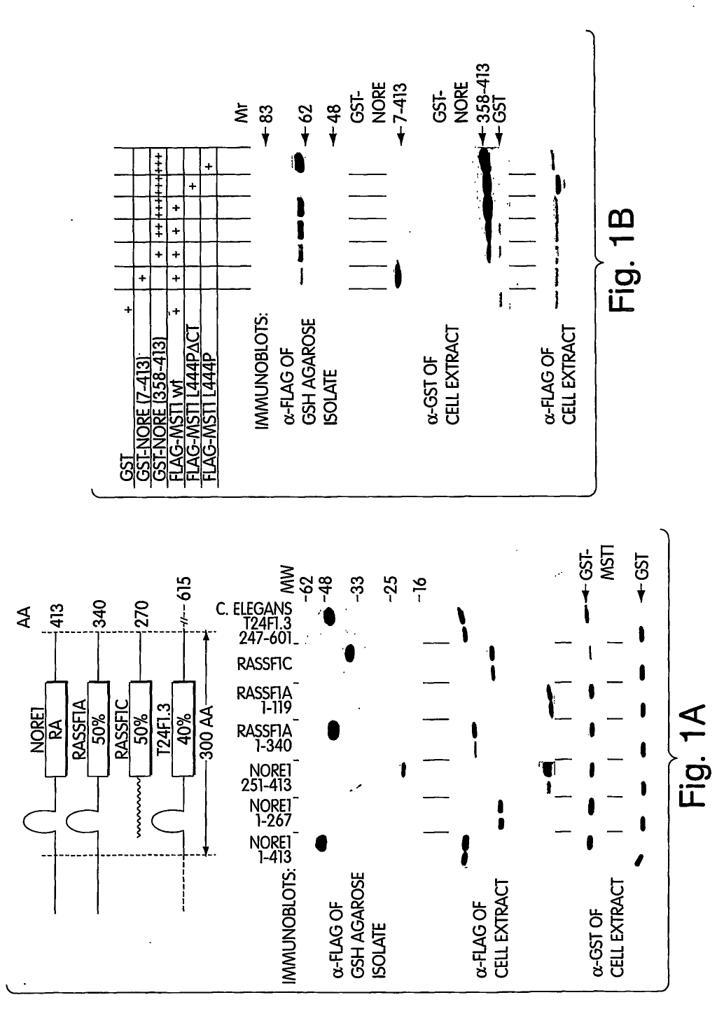

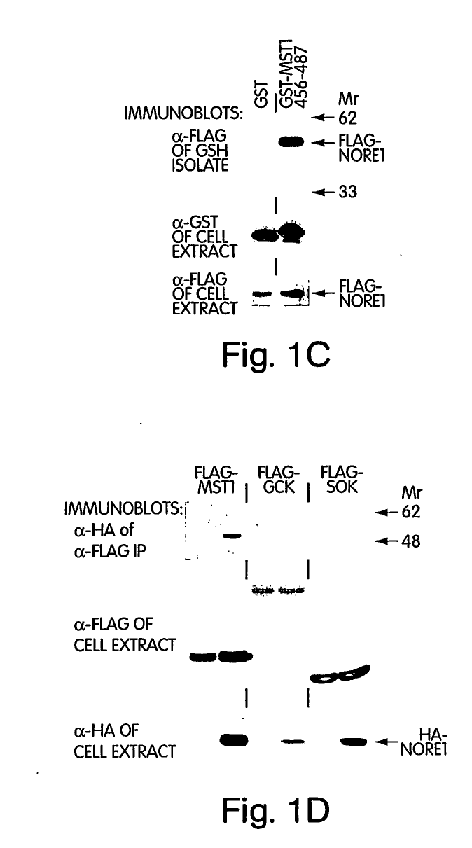

NORE is most closely related in structure to a family of human polypeptides encoded by the RASSF1 gene on Chr3p21 (Lerman et al., Cancer Res., 60: 6116-6133 (2000); Dammann et al., Nat. Genet., 25: 315-319 (2000)). Five splice variants of RASSF1 have been reported encoding amino acid sequences ranging from 189 to 344 amino acids. The three longest RASSF1 isoforms, A, D, and E (encoding 340, 344 and 344 amino acid polypeptides, respectively) each contain a central C1 zinc finger upstream of an RA domain, and are approximately 50% identical (75% similar) in amino acid sequence to the carboxy terminal 300 amino acids of NORE, diverging entirely from NORE only over their amino terminal fifty amino acids, which lack the proline rich segments present in NORE (see, e.g., FIG. 1A). An amino acid sequence for RASSF1A is shown in SEQ ID NO:3 and a nucleotide sequence encoding RASSF1A is shown in SEQ ID NO:4. The 270 amino acid long RASSF1C isoform is identical over its carboxy terminal 221 amino acids to the longer RASSF1 polypeptides but contains a unique 49 amino acid N-terminal segment; RASSF1C thus lacks the C1 zinc finger motif but contains the RA domain. An amino acid sequence for RASSF1C is shown in SEQ ID NO:5 and a nucleotide sequence encoding RASSF1C is shown in SEQ ID NO:6. The C. elegans gene product T24 F1.3 is a 615 amino acid polypeptide (SEQ ID NO:7) containing a unique amino terminal segment, a central C1 zinc finger (amino acids 165-214 of SEQ ID NO:7), a putative RA domain (amino acids 396-495 of SEQ ID NO:7), and a carboxy terminal extension of 65 amino acids relative to NORE and RASSF1A; the carboxy terminal 300 amino acids of the latter two polypeptides are each about 40% identical (70% similar) in amino acid sequence to the central segment of T24F1.3 (see, e.g., FIG. 1A), suggesting that T24F1.3, which contains the C1 and RA domains, is a common precursor to these two mammalian polypeptides. A nucleotide sequence encoding T24F1.3 is shown in SEQ ID NO:8.

The chromosomal segment encompassing the RASSF1 gene (3p21.3) exhibits loss of homozygosity in 90% of small cell lung cancers (SCLC), and in 50-80% non-small cell lung cancers (NSCLC) (Lerman et al., Cancer Res., 60: 6116-6133 (2000); Dammann et al., Nat. Genet., 25: 315-319 (2000)), breast cancers (Burbee et al., J. Natl. Cancer Inst., 93: 691-699 (2001); Dammann et al., Cancer Res., 61: 3105-3109 (2001)), clear cell renal cancers (Dreijerink et al., Proc. Natl. Acad. Sci. USA, 98: 7504-7509 (2001)), and several other solid tumors. Moreover, the expression of the longer isoforms, specifically RASSF1A, is selectively extinguished in all SCLC-derived cell lines examined and in a variety of other tumor-derived cell lines, through mutation or more frequently, methylation of the RASSF1A promoter (Dammann et al., Nat. Genet., 25: 315-319 (2000); Burbee et al., J. Natl. Cancer Inst., 93: 691-699 (2001); Dammann et al., Cancer Res., 61: 3105-3109 (2001); Dreijerink et al., Proc. Natl. Acad. Sci. USA, 98: 7504-7509 (2001)). Selective re-expression of the RASSF1A polypeptide suppresses the growth in vitro and the tumorigenicity in vivo of these cancer cell lines (Dammann et al., Nat. Genet., 25: 315-319 (2000); Burbee et al., J. Natl. Cancer Inst., 93: 691-699 (2001); Dammann et al., Cancer Res., 61: 3105-3109 (2001)).

NORE, RASSF1A, and T24F1.3 Bind to the Pro-Apoptotic Protein Kinase MST1

The tumor suppressor function attributed to RASSF1A together with the presence of an RA domain homologous to that in NORE raised the possibility that these polypeptides might participate in Ras-induced apoptosis. Surprisingly, in comparison to NORE, neither RASSF1A nor the C. elegans polypeptide T24F1.3 exhibited any significant ability to bind directly to RasG12V or several related GTPases, as determined quantitatively in a yeast two-hybrid assay, by cotransfection in mammalian cells (Ortiz-Vega et al., Oncogene, (2001)) or by binding in vitro (see, Examples below). Thus, while RASSF1 is unlikely to be a direct mediator of Ras function, the similarity between NORE, RASSF1A, and the central domain of T24F1.3A suggests that some evolutionarily conserved function was shared by these proteins. Accordingly, as described herein, a yeast two-hybrid expression cloning was employed in an effort to identify proteins that bound to all three NORE-related polypeptides. In screening cDNA libraries with either NORE or RASSF1A (i.e., using NORE and RASSF1A“baits)”, several candidates were found that interacted with both baits. However, only one also bound to T24F1.3, a murine cDNA encoding the Ste20-relatedprotein kinase MST1 (FIG. 1A). This result together with a prior demonstration that MST1 can act as a proapoptotic agent suggested further study for an evolutionarily conserved interaction. Accordingly, the interaction between NORE and MST1 was studied further in view of the clear-cut relationship between Ras and NORE.

Murine MST1 is a 487 amino acid polypeptide that migrates near 60 kilodaltons (kDa) on SDS-polyacrylamide gel electrophoresis (SDS-PAGE) (Creasy et al., J. Biol. Chem. 270: 21695-21700 (1995); Taylor et al., Proc. Natl. Acad. Sci. USA, 93: 10099-10104 (1996)). MST1 contains an amino terminal kinase catalytic domain (amino acids 30-281 of SEQ ID NO:9), which is 88% identical to MST2, and a noncatalytic carboxy terminal tail; indicative of a Group II GC kinase (Dan et al., Trends Cell Biol., 11: 220-230 (2001)). An amino acid sequence for MST1 is shown in SEQ ID NO:9 and a nucleotide sequence encoding MST1 is shown in SEQ ID NO:10. However, little has been known of the physiologic regulation of MST1. As for targets downstream of MST1, no effect on ERK 1/2 activity has been observed, and activation of coexpressed SAPK/JNK or p38 has been observed in some reports (Graves et al., EMBO J., 17: 2224-2234 (1998)), but not others (Creasy et al., J. Biol. Chem. 270: 21695-21700 (1995); Lee et al., Oncogene, 16: 3029-3037 (1998)). In contrast, overexpression of MST1 or a polypeptide portion of MST1 (i.e., amino acids 1-330 of SEQ ID NO:9) (but not kinase-inactive MST1) results in apoptosis in several cell backgrounds.

As described in the Examples below, FLAG-tagged versions of NORE, RASSF1A, RASSF1C, and T24F1.3 each bind specifically with GST-MST1 during transient expression in COS-7 cells (FIG. 1A). The binding site for MST1 is located on the carboxy terminal portions of NORE (FIG. 1A and 1B) and RASSF1A (FIG. 1A). The site on NORE responsible for binding MST1 was mapped more closely and was shown to be the carboxy terminal segment, distal to the RA domain. Thus, fusion of the NORE segment (358-413) (corresponding to amino acids 358-413 of SEQ ID NO:1) onto GST is sufficient to confer the binding of full-length MST1 (FIG. 1B). The MST1 segment responsible for binding NORE is at the MST1 carboxy terminus. Deletion of MST1 amino acids 449-487 of SEQ ID NO:9 abolishes binding to the GST-NORE (358-413) fusion polypeptide (FIG. 1B), whereas fusion of the MST1 carboxy terminal 32 amino acid segment (456-487) (i.e., amino acids 456-487 of SEQ ID NO:9) to GST is sufficient to enable the specific binding of FLAG-NORE (FIG. 1C). Moreover, MST1 dimerization is not required for association to NORE, as the dimerization-deficieint MST1 (L444P) mutant exhibits unimpaired binding to GST-NORE (358-413) fusion polypeptide (see, FIG. 1B).

The specificity of NORE binding to MST1 was evaluated by the examination of NORE binding to other representative GC kinase homologs (Dan et al., Trends Cell Biol., 11: 220-230 (2001)); thus, GCK or SOK1 do not bind to NORE (FIG. 1D). Also, NORE does bind MST2 (data not shown).

NORE and MST1 Exist as a Complex In Vivo

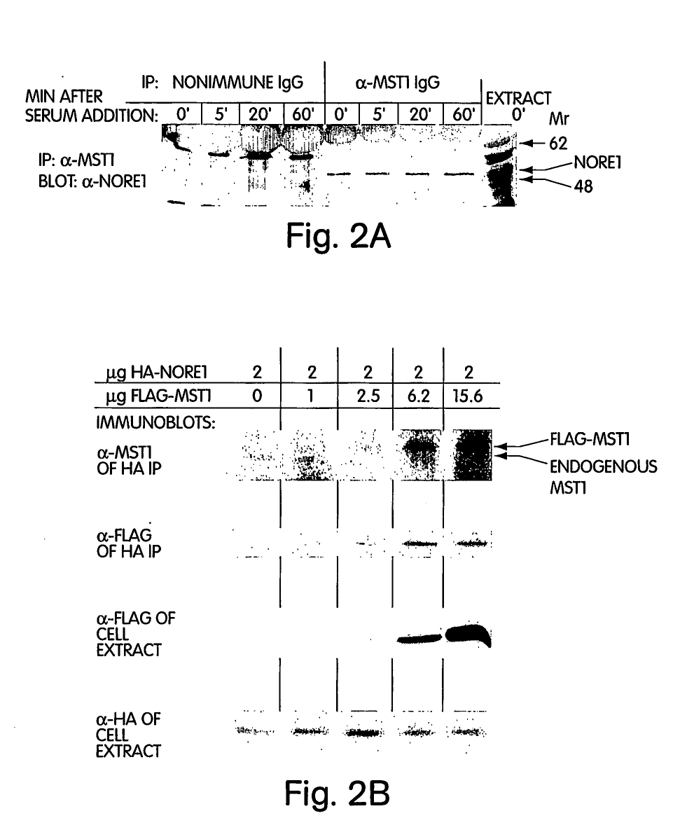

As shown previously, although KB cell extracts exhibit two major bands (at 46 kDa and 55 kDa) reactive on immunoblot with polyclonal anti-NORE antibody (FIG. 2A), only the 46 kDa band was recovered in anti-cRas (Y13-238) immunoprecipitates, and only after serum stimulation (Vavvas et al., J. Biol. Chem., 273: 5439-5442 (1998)); i.e., NORE can associate with Ras in a complex in vivo. As described in the Examples below, anti-MST1 immunoprecipitates prepared from KB cells also contained the 46 kDa NORE polypeptide whose presence was unaffected by serum deprivation or stimulation; i.e., MST1 can associate with NORE in a complex in vivo (see, FIG. 2A).

The polyclonal anti-NORE antibodies employed in this study did not enable reliable immunoprecipitation of endogenous NORE. Therefore, a recombinant hemagglutinin (HA)-tagged NORE fusion polypeptide was constructed and expressed by standard methods so that a more reliable, higher affinity, anti-HA (αHA) antibody could be employed to immunoprecipitate NORE polypeptide. The resulting HA immunoprecipitates could then be probed for endogenous MST1. The sensitivity of the detection system was further enhanced by constructing a recombinant FLAG-tagged MST1, which was simultaneously expressed in increasing amounts with the HA-NORE polypeptide (see Examples below). As seen in FIG. 2B, an immunoreactive band was observed in the anti-MST1 (αMST1) immunoblot of the HA-NORE immunoprecipitate that is identical in mobility to the MST1 band observed in the cell extract. Moreover, the increasing expression of the slightly larger FLAG-MST1 was accompanied by the appearance of increasing amounts of a slightly larger αMST1-reactive polypeptide in the HA-NORE immunoprecipitates, which ultimately displaced entirely the endogenous MST1 polypeptide. Thus, MST1 and NORE form a NORE-MST1 complex in vivo, which is constitutive (i.e., insensitive to serum withdrawal or addition) and saturable.

Active Ras Binds the NORE-MST1 Complex In Vivo

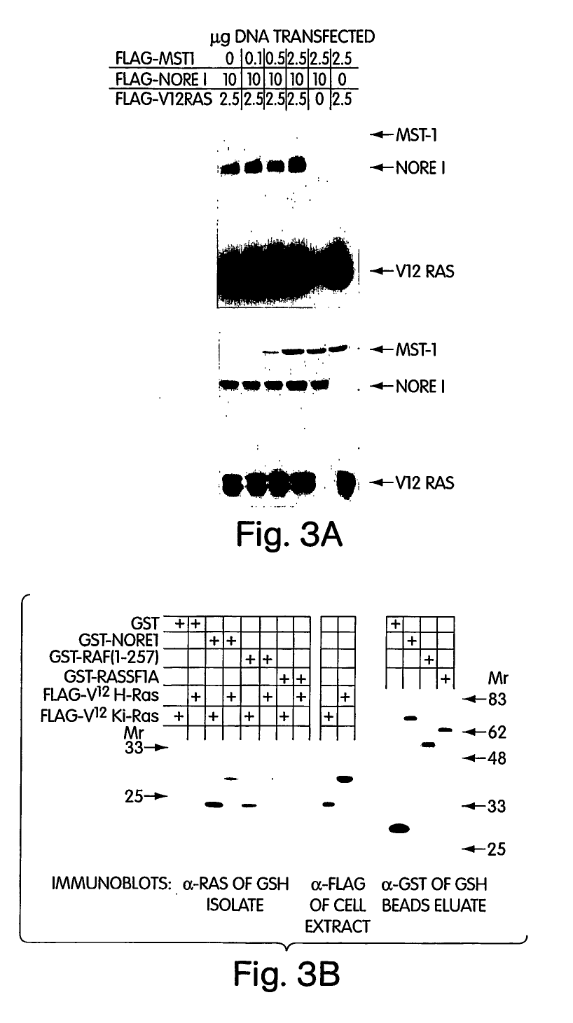

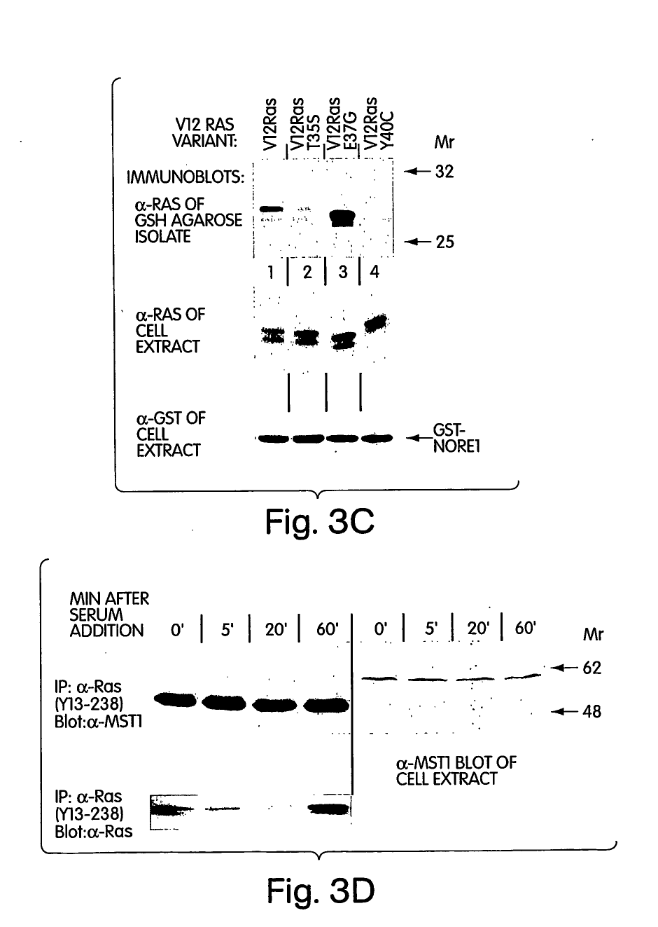

As noted above, NORE is known to bind Ras. Accordingly, it was important to determine whether the NORE-MST1 complex that exists in vivo also binds Ras in vivo. To test this possibility, a constant amount of a FLAG-tagged Ha-RasG12V was expressed with a constant level of FLAG-tagged NORE and increasing amounts of FLAG-tagged MST1. The FLAG-tagged HA-RasG12V was immunoprecipitated with the anti-Ras antibody Y13-238, and the initial cell extracts and the Ras immunoprecipitates were probed for FLAG-tagged polypetides (see, FIG. 3A). As seen in the upper panel in FIG. 3A, the FLAG-MST1 polypeptide was recovered in a RasG12V immunoprecipitate, but only in the presence of coexpressed NORE. Moreover, by comparing the intensities of FLAG-NORE and FLAG-MST1 in the cell extract to that of the Ras immunoprecipitate, it could be estimated that, in the presence of an excess of RasG12V, about 20% of both FLAG-NORE and coexpressed FLAG-MST1 are recovered in the RasG12V immunoprecipitate. Thus, active Ras is able to bind the NORE-MST1 complex. GST-NORE and GST Raf (1-257) each bind in vitro slightly better to Ki-RasG12V than to Ha-RasG12V, whereas GST RASSF1A binds neither Ras polypeptide (see, FIG. 3B).

Previously it was shown that Ras binds NORE through the Ras effector loop; mutation of Ras Asp38 to Ala or Asn abolishes Ras binding to NORE (Vavvas et al., J. Biol. Chem., 273: 5439-5442 (1998)). Here, the ability of several other Ha-RasG12V effector loop mutants to bind NORE during transient expression was examined (FIG. 3C). The mutation Ha-RasT35S greatly diminishes, and Y40C abolishes, the Ha-RasG12V-NORE interaction; conversely, the Ha-RasG12V, E37G mutant exhibits unimpaired (and perhaps enhanced) binding to NORE.

The ability of endogenous Ras to bind endogenous MST1 was evaluated in KB cell. Endogenous cRas was immunoprecipitated from cell extracts prepared from serum-deprived KB cells and at several times after serum addition; the anti-Ras antibody (Y13-238) immunoprecipitates were probed for MST1. A band of immunoreactive MST1 was observed in the cRas immunoprecipitates only after serum addition and persisted for up to 1 hour (see FIG. 3D); this result was similar to the serum-induced association of endogenous NORE with cRas reported previously (Vavvas et al., J. Biol. Chem., 273: 5439-5442 (1998)). Thus, serum-induced activation of endogenous Ras causes the recruitment of the NORE-MST1 complex to the Ras effector loop.

Membrane Recruitment of MST1 Promotes Its Apoptotic Action

The functional significance of the Ras-NORE-MST1 interaction to the pleiotypic array of cellular responses mediated by Ras was also examined. The immediate downstream targets of MST1 are unknown; however, it has been consistently observed that overexpression of recombinant MST1 results in apoptosis in a number of cell backgrounds (Graves et al., EMBO J., 17: 2224-2234 (1998); Lee et al., Oncogene, 16: 3029-3037 (1998); Graves et al., J. Biol. Chem., 276: 14909-14915 (2001)). Although the pathway by which MST1 promotes apoptosis is unknown, apoptosis was employed as a quantifiable measure of the efficacy of MST1 action in vivo.

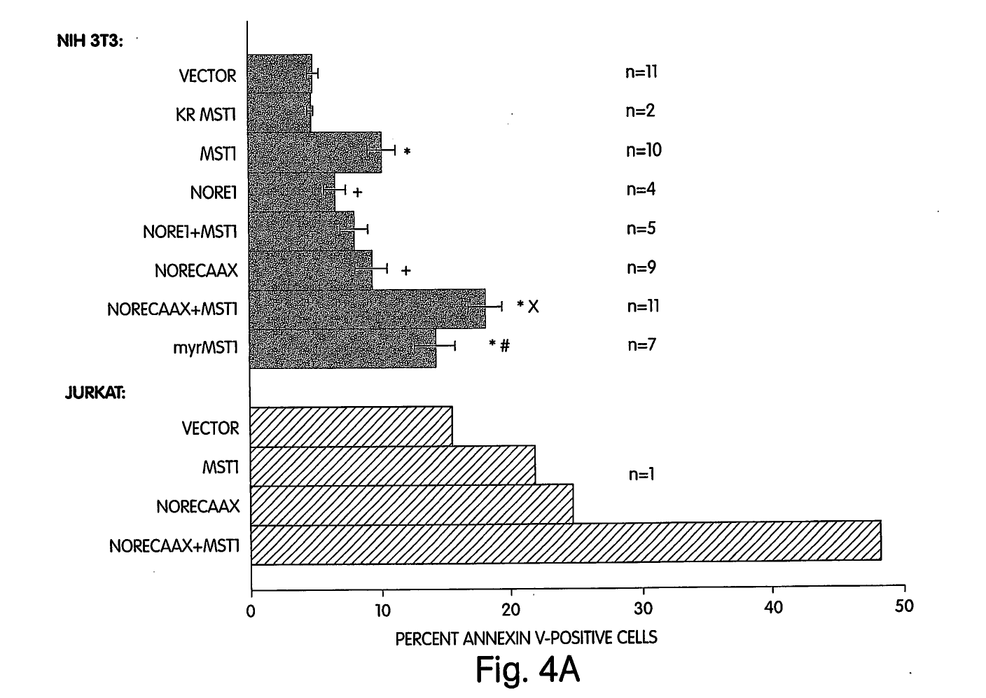

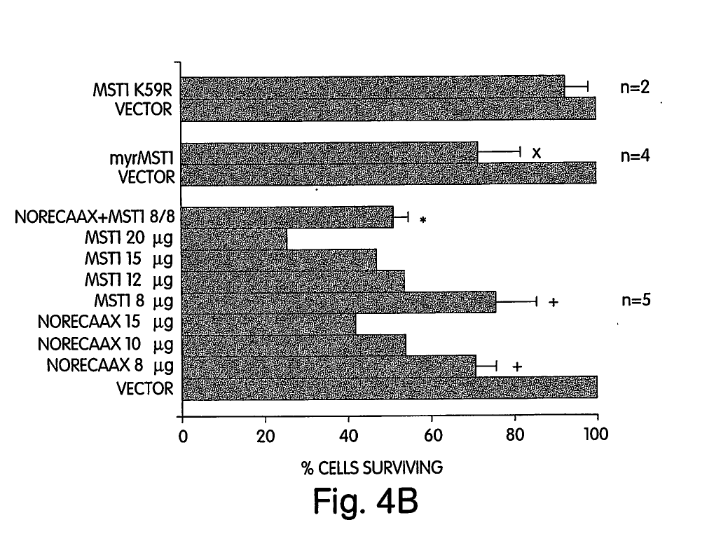

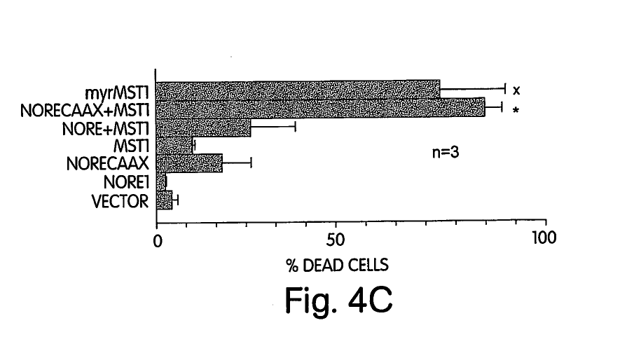

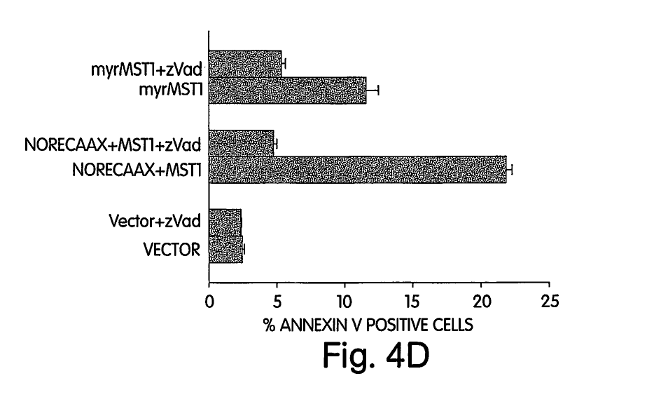

As expected from previous reports, transient expression of MST1 results in apoptosis in several cell types, measured either as an increase in annexin V surface binding (i.e., “staining”, see, FIG. 4A) or as a decrease in the percent survival of transfected cells (FIGS. 4B and 4C). An initial series of studies (described in the Examples below) was carried out in NIH3T3 cells (FIG. 4A); overexpression of MST1 gave a 2-fold increase in the fraction of cells staining with annexin V at 24 hours after transfection, whereas MST1 (KR) gave no alteration as compared to vector control. NORE alone caused a statistically significant but very small increase in annexin V staining of NIH3T3 cells, and the combined expression of NORE and MST1 yielded annexin V staining slightly (but not significantly) less than that seen with MST1 alone. By contrast, NORECAAX (NORE fused to a membrane association peptide) causes a doubling in the fraction of annexin V-positive cells, and coexpression of MST1 with NORECAAX resulted in an increase in the fraction of annexin V-stained cells that was significantly (about 50%) greater than the sum of the effects of MST1 and NORECAAX individually (FIG. 4A) and (in two experiments) comparable to the proapoptotic effect to Baxa (data not shown). The ability of NORECAAX and MST1 to promote annexin B staining was fully suppressed by inclusion of zVAD-FMK in the medium after transfection (FIG. 4D). A similar pattern of responses to MORE and NORECAAX, singly and in combination with MST1, was seen in Jurkat (FIG. 4B) and HEK293 cell (FIG. 4C). In both cells' backgrounds, MST1 (but not the inactive MSt1 KR mutant) and NORECAAX caused a dose-dependent (examined in Jurkat only) increase in apoptosis, and the combination of low doses of NORECAAX with MST1 caused additive (Jurkat) or superadditive MEK293) killing, which was suppressed by the presence of BV p35 CrmA plus BcIX_{L} or incubation with zVAD-FMK (data not shown).

The ability of NORECAAX to augment the response to MST1 suggested that the recruitment of MST1 to the membrane enhanced it apoptotic action. To examine this idea, the amino terminal myristoylation and polybasic segment of cSrc (“myr”; Reuther et al., Methods Enzymol., 327: 331-350 (2000)) was fused onto the amino terminus of MST1 (to form “myrMST1”, see, Examples below). Although myristoylation (or coexpression with excess NORECAAX) tends to decrease MST1 polypeptide expression per μg of MST1 cDNA in comparison to MST1 alone, the apoptotic efficacy (per μg DNA) of myrMST1 was about 2-fold greater than MST1 in NIH3T3 cells. In addition, side-by-side comparisons in NIH3T3 cells suggest that apoptosis induced by myrMST1 is about 50% less than by MST1 coexpressed with NORECAAX (FIG. 4A). In Jurkat cells, the efficacy of myrMST1 was only slightly greater than MST1 itself and much less than that of MST1 coexpressed with NORECAAX (FIG. 4B); in contrast, the proapoptotic efficacy of myrMST1 in HED293 cells (FIG. 4C) was markedly greater than MST1, and in three experiments, only slightly less than MST1 coexpressed with NORECAAX.

Thus, the apoptotic effect of MST1 is generally increased by membrane targeting, either by myristoylation, or most consistently by coexpression with a membrane-targeted variant of NORE.

RasG12V Promotes Apoptosis through a NORE-MST1 Complex

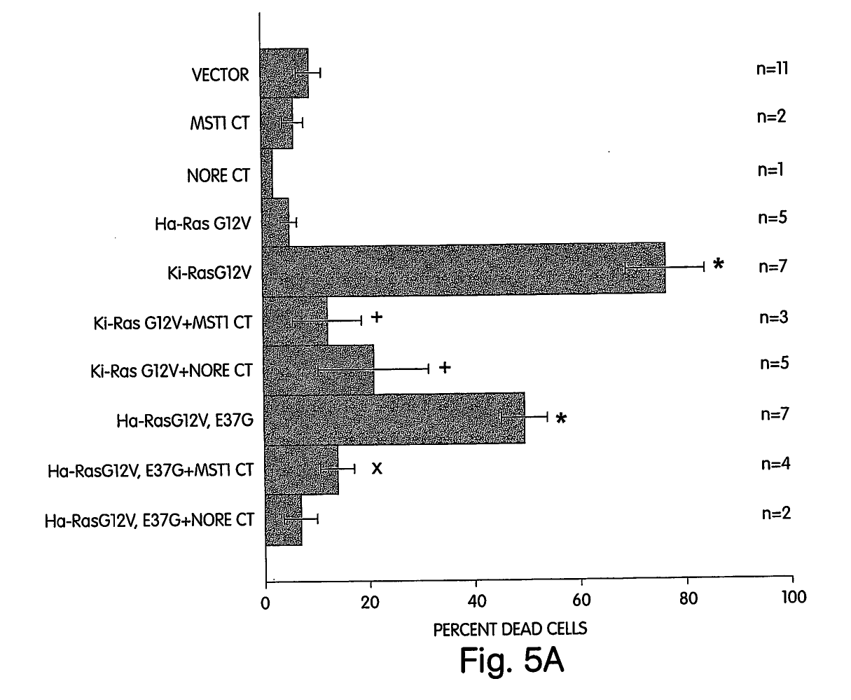

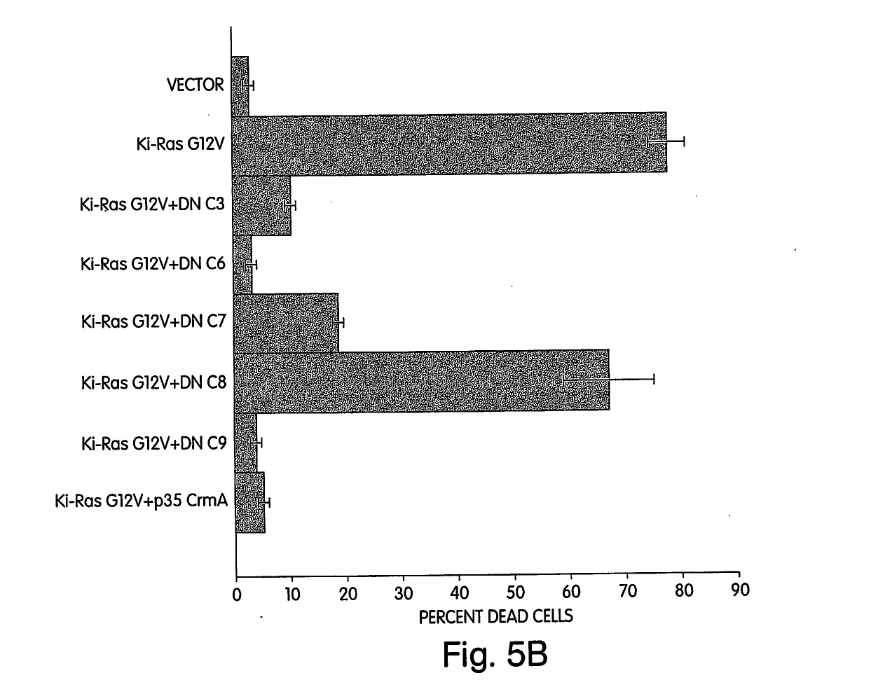

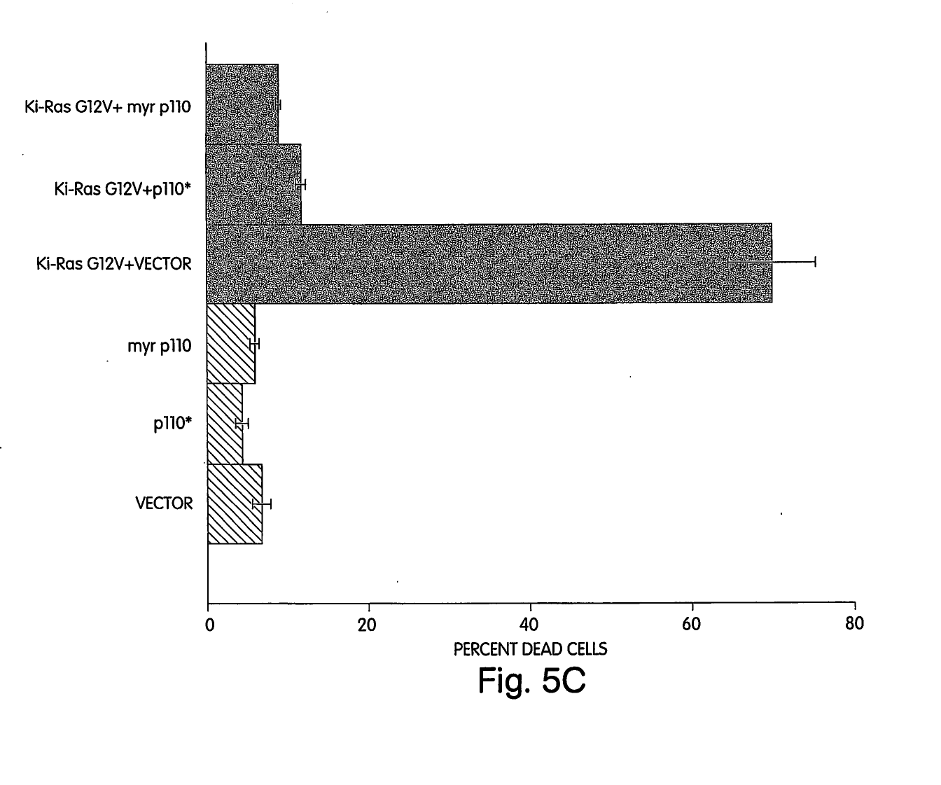

The ability of Ras to recruit a NORE-MST1 complex, coupled with the increased apoptotic efficacy of membrane-targeted MST1, is consistent with the view that the NORE-MST1 complex acts as a proapoptotic effector of constitutively active forms of Ras. This was tested using HEK293 cells, in view of the high-grade apoptosis observed with myrMSt1 or NORECAAX plus MST1 in this cell background. Overexpression of Ki-RasG12V in 293 cells efficiently induced cell death (FIG. 5A). In contrast, Ha-RasG12V gave no significant cell death, in spite of the consistently higher Ha-Ras polypeptide expression achieved when comparable levels of Ha-Ras and Ki-Ras DNA are transfected. The expression of Ki-RasG12V per se is sufficient to cause cell death, which becomes maximal at 48-72 hours; however, the time course of Ki-RasG12V-induced cell death is considerably accelerated by the addition after transfection of low amounts of proapoptotic agents like tamoxifen or staurosporine at concentrations below those capable of directly inducing apoptosis in HEK293 cells transfected with empty control vectors.

Although commonly considered interchangeable, a number of differences in the biologic responses to HaRasG12V and Ki-RasG12VB have been observed previously (Ellis et al., Cell Signal, 12: 425-434 (2000)). Of particular note (42), HaRasG12V is considerably (7-fold) more effective in activating PI 3-kinase than is Ki-RasG12V. Reasoning that the greater ability of Ha-RasG12V to activate this major antiapoptotic effector may suppress the expression of a concomitant proapoptotic outflow, we examined the proapoptotic effect of the Ha-Ras effector loop mutant G12V, E37G, was examined. The Ha-Ras effector loop mutant G12V, E37G, is unable to bind PI-3 kinase and Raf, yet remains competent to bind NORE (see, FIG. 3C); Ha-RasG12V, E37G elicits substantial cell death in 293 cells, exhibiting perhaps ⅔ the potency of Ki-RasG12V (see, FIG. 5A). The cell death elicited by Ki-RasG12V and Ha-RasG12V, E37G is inhibited by coexpression with the baculoviral antipoptotic protein p35 CrmA (FIG. 5B; other data not shown) or by preincubation with zVAD-FMK (data not shown). Moreover, although the appearance of HEK293 cells overexpressing Ha-RasG12V did not differ significantly from GFP controls, expression of Ki-RasG12V and Ha-RasG12V, E37G is accompanied by changes in cell morphology characteristic of apoptotic cell death, i.e., prominent membrane blebbing, loss of cytoarchitecture, and nuclear and cytoplasmic fragmentation resulting in apoptotic bodies. In addition, coexpression of Ki-RasG12V with dominant inhibitory mutant of caspase 3, 6, 7, and 9 each substantially suppressed the Ki-RasG12V-induced cell death, whereas dominant inhibitory caspase 8 had no effect (FIG. 5B). Thus, the cell death elicited by Ki-RasG12V and Ha-RasG12V, E37G in HEK293 cells reflects a caspase-mediated process probably occurring primarily through activation of the “intrinsic” (i.e., TNF receptor superfamily and caspase 8-idependent) pathway. The apoptosis induced by Ki-RasG12V is potently suppressed by coexpression with constitutively active variants of the PI 3-kinase p110 catalytic subunit (FIG. 5C).

Whether the ability of Ki-RasG12V and RasG12V, E37G to bind the NORE-MST1 complex was critical to their proapoptotic action was also examined. This was evaluated by examining whether overexpression of the segment of NORE responsible for binding MST1 (introduced as GST-NORE[358-413]) or the carboxy-terminal noncatalytic segment of MST1 (introduced as FLAG-MST1[307-487]) were each able to interfere specifically with apoptosis induced by Ki-RasG12V and Ha-RasG12V, E37G (FIG. 5A). Neither GST-NORE (358-413) nor FLAG-MST1(307-487) expressed alone altered cell survival; conversely, expression of either fragment inhibited nearly completely the ability of Ki-RasG12V and Ha-RasG12V, E37G to induce apoptosis. The inhibitory action of the NORE and MST1 carboxy termini was specific for apoptosis initiated by RasG12V; neither the GST-NORE fusion nor the MST1 carboxy terminal segment inhibited Fas-induced apoptosis in the Jurkat cell background (data not shown). Thus, sequestration of MST1 (by GST-NORE[358-413]) or expression of the MST1 noncatalytic segment, which contains autoinhibitory, dimerization, and NORE binding domains, each inhibits selectively the ability of Ki-RasG12V and Ha-RasG12V, E37G to promote apoptosis.

MST1 Kinase Activity

Whether NORE and/or RasG12V regulate MST1 kinase activity was also examined. The readdition of serum to 293 of KB cells deprived overnight did not alter the activity of immunoprecipitated endogenous MST1. Cotransfection of MST1 (and in one experiment, MST2) with an excess of NORE was accompanied by an increase in MST1 expression; however, the specific activity of MST1 assayed in vitro was suppressed by up to 80%. Serum readdition in the presence of coexpressed NORE did not cause significant alteration in the activity of recombinant MST1 or in its mobility on SDS-PAGE. The coexpression of Ha- or Ki-RasG12V with MST1, or with a NORE-MST1 complex, gave no consistent change of MST1 activity. The apparent suppression of recombinant MST1 activity by coexpression with NORE and the absence of RasG12V-induced alteration in the activity of recombinant MST1, as well as the observation that serum induces the binding of an endogenous NORE-MST1 complex to endogenous Ras without detectably altering the catalytic activity of MST1 signaling primarily by its recruitment of MST1 to a site critical for MST1 action.

MST1 resides predominantly in the cytoplasm. A portion of MST1 is constitutively bound to NORE1 and the RASSF1A and RASSF1C polypeptides. The proapoptotic activity of MST1 is greatly enhanced by targeting MST1 to the plasma membrane. In the case of recombinant MST1, this was shown by fusion of a myristoylation motif directly to the MST1 amino terminus, or by coexpressing wild type MST1 with NORE-CAAX, a variant of NORE genetically engineered to be constitutively associated with the plasma membrane (see, Khokhlatchev et al., Curr. Biol., 12(4): 253-265 (2002)). In the case of endogenous NORE, this translocation is accomplished by the association of NORE with activated, GTP-bound Ras. The RASSF1A polypeptide (but not the RASSF1C isoform) is a tumor surpressor whose expression is extinguished in a large fraction of human lung, breast, and other cancers (see, Pfeifer et al., Biol. Chem., 383(6): 907-914 (2002)). The ability of RASSF1A to act as a tumor surpressor is dependent in part on its ability to bind MST1 and transport MST1 to the surface membrane (and perhaps other sites of activation). Unlike NORE, neither RASSF1A nor RASSF1C can bind directly to active Ras. Nevertheless, RASSF1A (but not RASSF1C) can readily form dimers with NORE, and this RASSF1A-NORE heterodimer can bind to Ras through NORE (see, Ortiz-Vega et al., Onicogene, 21(9): 1381-90 (2002)). Therefore, reexpression of RASSF1A can augment the amount of MST1 at the plasma membrane and promote apoptosis of tumor cells expressing active Ras or oncogenes that activate Ras.

Methods of the Invention

The elucidation of a particular Ras-mediated apoptotic pathway, major effector protein components, and the critical interactions of such component effectors, as described herein, provides the basis of methods of screening, identifying, or otherwise evaluating one or more compounds as proapoptotic drugs that have a specific target of action. The ability to switch on a specific pathway of apoptosis may be particularly useful in conditions where it is desirable to arrest cell division, such as in various forms of cancer. Moreover, a compound identified according to this invention may be particularly compatible in treatment protocols currently employing other anti-cancer drugs that are known to or likely to act at other targets in a cancer cell.

Accordingly, the invention provides methods of identifying or evaluating a compound that stimulates formation of a complex comprising NORE and/or RASSF1A, MST1 (or MST2), and Ras to induce a Ras-mediated apoptosis. Formation of such complexes may be tested by contacting cells (in vivo) or even in a mixture (including, but not limited to, defined mixtures, extracts of cells, and combinations thereof) of effector proteins (in vitro) with a test compound and measuring the level of complex formation relative to the level in the absence of the compound. Cells employed in methods of the invention must have the functional genetic information to express the effector proteins (e.g., NORE1, MST1 or MST2, RASSF1A) and a Ras protein, as needed, that will provide a particular apoptotic pathway described herein. For example, the functional genetic information necessary for expression of effector proteins and Ras may be part of the original genome of the cells (i.e., endogenous to the cells) and/or provided by recombinant methods (i.e., exogenously provided to the cells). Cells may be assayed directly for expression of a particular protein or protein complex by immunostaining with appropriate labeled antibodies. In some cases, when cells are employed in the methods of the invention, an extract of the cells may be prepared by any of a variety of standard methods known in the art, and preferably such methods do not disrupt non-covalently associated protein complexes. In a method of the invention, the level of a complex formed in cells or a mixture of effector proteins contacted with a test compound may be compared to the level of complex formed in the absence of the test compound. A particularly preferred method for assaying for the formation of a complex comprises immunoprecipitation or immunostaining with an antibody to a particular component effector protein, followed by further analysis for other effector protein species using antibodies that specifically bind those other component species.

A preferred method of identifying or evaluating a compound that induces a Ras-mediated pathway of apoptosis in eukaryotic cells comprises:
a) providing eukaryotic cells having the genetic information for expressing a NORE protein, an MST1 or MST2 protein kinase, a Ras protein, and, optionally, an RASSF1A protein;
b) contacting said eukaryotic cells with a test compound; and
c) assaying said eukaryotic cells contacted with said test compound for an increased level of formation of a complex comprising NORE, MST1 or MST2, and Ras proteins or, optionally, of RASSF1A, MST1 or MST2, and Ras proteins, compared to the level of formation of said complex in cells not contacted with said test compound;
   wherein an increase in the level of said complex formation indicates that said test compound induces a Ras-mediated apoptotic pathway in eukaryotic cells.

Another method of identifying or evaluating a compound that stimulates a Ras-mediated pathway of apoptosis in eukaryotic cells comprises:
a) providing eukaryotic cells having the genetic information for expressing a NORE protein;
b) contacting said eukaryotic cells with a test compound; and
c) assaying said eukaryotic cells contacted with said test compound for increased expression of NORE protein or increased synthesis of NORE-encoding mRNA compared to cells not contacted with said compound;
   wherein an increase in NORE protein expression or in synthesis of NORE-encoding mRNA indicates that said test compound induces a Ras-mediated pathway of apoptosis in eukaryotic cells.

Another method of identifying or evaluating a compound that stimulates a Ras-mediated pathway of apoptosis in eukaryotic cells comprises:
a) providing eukaryotic cells having the genetic information for expressing an RASSF1A protein;
b) contacting said eukaryotic cells with a test compound; and
c) assaying said eukaryotic cells contacted with said test compound for increased expression of RASSF1A protein or increased synthesis of RASSF1A-encoding mRNA compared to cells not contacted with said test compound;
   wherein an increase in RASSF1A protein expression or in synthesis of RASSF1A-encoding mRNA indicates that said test compound stimulates a Ras-mediated pathway of apoptosis in eukaryotic cells.

Still another method of identify or evaluating a compound that stimulates a pathway of apoptosis in eukaryotic cells comprises:
a) providing eukaryotic cells having the genetic information for expressing an MST1 or MST2 protein kinase;
b) contacting said eukaryotic cells with a test compound;
c) assaying said eukaryotic cells contacted with said test compound for increased expression of MST1 or MST2 protein or increased synthesis of MST1 or MST2-encoding mRNA compared to cells not contacted with said compound;
   wherein an increase in MST1 or MST2 expression or in synthesis of MST1 or MST2-encoding mRNA indicates that said test compound stimulates a pathway of apoptosis in eukaryotic cells.

Assays for screening or evaluating a test compound according to the invention may employ any eukaryotic cell, including but not limited to, any Ras-transformed eukaryotic cell or any other mammalian cell, that possesses the natural or recombinant genetic information necessary to express the various critical effector proteins (e.g., MST1, MST2, NORE, and/or RASSF1A) that associate in a complex to stimulate or induce an apoptotic pathway described herein. Preferably, a cell expresses a particular effector protein, whose level of expression is monitored in an assay described herein, at a level equal to or lower than the level in the wild type form of the cell, so that a difference in expression of the effector protein in the presence and absence of a test compound may be readily detected. As compounds are usually screened or evaluated as candidates for development and use as anti-cancer drugs in humans, the methods of the invention preferably employ cells of a human cancer (tumor) cell line. Such human cancer cells useful in the methods described herein include, but are not limited to, lung cancer cells (e.g., cells of the A549 lung cancer cell line), melanoma cancer cells (e.g., cells of the G361 human melanoma cell line), myeloma cancer cells, leukemia cancer cells, teratoma cancer cells, kidney cancer cells, brain cancer cells, bone cancer cells, bladder cancer cells, epithelial cancer cells, breast cancer cells, colon cancer cells, testicular cancer cells, and prostate cancer cells. It is understood that any compound identified or assessed as capable of inducing apoptosis in a particular cell employed in a method of the invention will usually be further tested for its ability to induce apoptosis in other types of cancer cells as well.

An alternative method of the invention for identifying or evaluating a compound that stimulates or induces an apoptotic pathway may comprise providing a eukaryotic cell that expresses a fusion protein, wherein the fusion protein comprises a portion of the amino acid sequence of a particular effector protein linked in frame to an epitope tag sequence (e.g., HA, FLAG, c-myc, and the like) for which high affinity antibodies are readily available commercially or readily obtained by standard antibody production protocols. In this way, the expression level of a fusion protein is monitored instead of the corresponding full-length effector protein molecule. The availability of high affinity antibodies for a particular epitope tag may provide a more sensitive, more convenient, and/or more reproducible assay to detect a change in effector protein expression. DNA molecules encoding such fusion proteins are readily made by recombinant DNA methods or polymerase chain reaction (PCR) methods available in the art. It is important that a DNA molecule encoding a particular fusion protein be operatively linked to an appropriate promoter that promotes transcription of the corresponding full-length effector protein or of another gene that participates in a particular apoptotic pathway described herein. Some representative examples of cells expressing such fusion proteins have been employed in studies described herein and in the Figures.

The assays to identify or evaluate a compound according to the invention may be carried on one or more compounds, as well as in any of a variety of formats that permit one or multiple compounds to be assessed simultaneously. For example, such formats may employ culture or assay dishes containing multiple wells, wherein each well provides a single culture of cells for testing a single compound. The efficiency of such a multi-well format may be further enhanced by employing an automated or semi-automated apparatus to test and/or assess the effect of multiple compounds simultaneously.

The level of expression of a particular effector protein or complex in a method of the invention may be determined using various methods available in the art including, but not limited to, measurement of levels of effector protein molecules and/or measurement of levels of mRNA transcripts encoding particular effector proteins.

An antibody that binds with specificity to an effector protein, a Ras protein, or an epitope tagged version thereof, as described herein, is particularly useful in the methods of the invention as there is a wide variety of immunoassay methods known in the art for detecting an antibody bound to its cognate antigen, and such immunoassay methods are readily adapted for use in the methods of the invention. Such immunoassay methods include, but are not limited to, immunoprecipitation (including co-precipitations), immunoblotting, enzyme-linked immunosorbent assay (ELISA), fluorescent antibody cell sorter (FACS) detection, affinity particles containing adsorbed or conjugated antibody molecules, and combinations thereof The terms “antibody”, “antibody molecule”, and similar terms, are understood to encompass polypeptide or portion thereof that specifically binds to an antigen, whether produced naturally, synthetically, or semi-synthetically, which possesses an antigenic binding domain formed by an immunoglobulin variable light chain region (V_{L}), or portion thereof, and/or an immunoglobulin variable heavy chain region or domain (V_{H}), or portion thereof. The term also covers any polypeptide or protein molecule that has an antigen binding domain which is identical, or homologous to, an antibody binding domain of an antibody immunoglobulin. Examples of an antibody molecule, as used and understood herein, include any of the well known classes of immunoglobulins (e.g., IgG, Ighl, IgA, IgE, IgD) and their isotypes; fragments of immunoglobulins that comprise an antigen binding domain, such as Fab or F(ab′)₂ molecules; Fv (variable regions), single chain antibody (scFv) molecules; double scFv molecules; heavy chain antibodies; single domain antibody (dAb) molecules; Fd molecules; and diabody molecules (see, e.g., Holliger et al., Proc. Natl. Acad. Sci. USA, 90: 6444-6448 (1993)). Furthermore, antibodies useful in the invention may be polyclonal (e.g., antiserum obtained from a rabbit, mouse, rat, or goat inoculated with an antigenic compound) or monoclonal (e.g., using standard hybridoma technology). Antibodies useful in the invention may be obtained commercially or by following standard immunological protocols known in the art. For example, as employed in studies described herein, an anti-MST1 antibody is commercially available (e.g., Zymed Laboratories, Inc., South San Francisco, Calif.), and a polyclonal rabbit anti-NORE1 antibody has been described (see, Vavvas et al., J. Biol. Chem., 273: 5439-5442 (1998)). An antibody useful in the invention may also include “affinity maturation” antibodies in which the affinity of an antibody (parent molecule) that binds an effector protein or a Ras protein has been enhanced by further mutation or selection by methods known in the art, such as phage display.

For a method of the invention employing a cell that expresses a fusion protein comprising a portion of a protein (e.g., an effector protein of interest) and a portion comprising one or more epitope tags (e.g., HA, FLAG, c-myc, and the like), high affinity antibodies are commercially available or readily produced to the epitope tag sequence. The availability of high affinity antibodies to such epitope tags may provide a particularly sensitive and reproducible assay for detecting compounds that stimulate expression of the corresponding full-length protein.

An antibody to a critical effector protein, a Ras protein, or an epitope tagged version thereof, as described above, may also be used in methods of the invention to detect formation of complexes that are critical to stimulate or induce apoptosis in a cell. For example, proteins in a cell or mixture may be contacted or mixed with an antibody that binds and detects a particular effector protein species to obtain an immunoprecipitate. The resulting immunoprecipitate could then be further analyzed for the presence of other protein species of interest (e.g., other effector proteins or a Ras protein) using antibodies to those other protein species that may have co-precipitated (see, e.g., FIG. 1A-1D; 2A-2B; and 3A-3D). Such co-precipitation with antibodies may also be used to distinguish or measure levels of formation of NORE homodimers and NORE-RASSF1A heterodimers of the complexes described herein.

Expression levels of a particular protein of interest (e.g., a particular effector protein) may also be determined by measuring levels of mRNA transcripts that encode that particular protein in a cell. Such methods are accurate provided there is no defect in the translation machinery of the cell. Levels of mRNA encoding a particular polypeptide are readily detected by methods available in the art, e.g., standard Northern blotting or reverse transcriptase polymerase chain reaction (RT PCR) procedures known in the art Preferably, an RT PCR protocol is employed owing to the significantly higher sensitivity over prior methods. As used in all PCR protocol, primer sequences employed in an RT PCR procedure to detect an mRNA encoding a particular protein may be designed from known 5′ and 3′ nucleotide sequences of the mRNA or corresponding cDNA.

Additional embodiments and features of the invention will be apparent from the following non-limiting examples.

### EXAMPLES

Materials and Methods for Studying Recruitment of MST1 to Induce Apoptosis in Ras-Transformed Cells.

DNA Constructs and Manipulations

Mouse MST1 cDNA was kindly provided by Dr. Leonard Zon (Children's Hospital, Boston, Mass.). cDNAs for human GCK and SOK protein kinases were kindly provided by Dr. John Kyriakis (Massachusetts General Hospital, Boston). cDNAs for human V12 Ha-Ras effector loop mutants E37G, T25S and Y40C were kindly provided by Dr. Michael White (Southwestern Medical Center, Dallas, Tex.). RASSF1A and RASSF1C cDNAs were kindly provided by Dr. Gerd Pfeifer (Beckman Research Institute, City of Hope, Calif.). cDNAs encoding catalytically inactive caspase 3, 6, 7, 8, and 9 were provided by Dr. Dale Bredesen and constructed as described by Lu et al. (Nature, 6: 397-404 (2000)). The baculoviral p35 caspase inhibitor CrmA, encoded in pCS2+, was provided by Dr. Vincent Cryns. C. elegans cDNA encoding amino acids 247-601 of the 724F1.3 gene product (i.e., amino acids 247-601 of SEQ ID NO:7) was kindly provided by Dr. Y. Kohara (Japan). A myc-tagged, constitutively active PI-3 kinase, p110* was prepared from the PI-3K p110* construct described previously (see, Hu et al., Science, 268: 100-102 (1995)). An amino acid sequence of a myc-tag is shown in SEQ ID NO:11. Human MST2 cDNA was obtained by PCR on a placental library cDNA with the primers corresponding to the 5′ and 3′ ends of the full-length cDNA. An amino acid sequence for active Ki-Ras(G12V) is shown in SEQ ID NO:12 and a nucleotide sequence encoding Ki-Ras(G12V) is shown in SEQ ID NO:13. An amino acid sequence for active Ha-Ras(G12V) is shown in SEQ ID NO:14 and a nucleotide sequence encoding Ha-Ras(G12V) is shown in SEQ ID NO:15.

Manipulationas of cDNAs were performed using the standard molecular biology techniques (Sarnbrook et al., Molecular Cloning: A Laboratory Manual (third edition) (Cold Spring Harbor, Cold Spring Harbor Laboratory Press, 2001)). Full-length and truncated NORE1, RASSF1A, RASSF1C, T24F1.3, MST1 and MST2 cDNAs, as well as Ki-RasG12VHa-RasG12V and its effector loop mutants were subdloned into pCMV5 FLAG vector in-frame downstream of the FLAG epitope (Met Asp Tyr Lys Asp Asp Asp Asp Lys Asn Ser Ala) (SEQ ID NO:16). For some constructs, FLAG epitope in pCMV5 was replaced with the influenza virus hemagglutinin epitope (HA, Met Tyr Pro Tyr Asp Val Pro Asp Tyr Ala) (SEQ ID NO:17) by excising the FLAG epitope and promoter region from pCMV FLAG plasmid with SacI and EcoRI enzymes and replacing it with the synthetic oligonucleotide coding for the promoter region followed by the HA epitope in-frame with the downstream sequences. The same approach was used for creation of the myristoylated MST1 and NORE1; the FLAG tag of the pCMV 5 was replaced with the “myr” peptide, which is a c-Src myristoylation and polybasic segment (Met Gly Ser Ser Lys Ser Lys Pro Lys Asp Pro Ser Gln Arg Arg Arg) (SEQ ID NO:18) followed by four glycine residues and an inframe FLAG tag. The same approach was used to construct the myristoylated PI-3 kinase catalytic subunit (myr-p110). The p110* was digested with BamnH1, releasing a 2.8 kilobases (kb) insert encoding full-length p110 with a carboxy terminal myc epitope. This was inserted in frame downstream of the c-Src myristoylation and polybasic segment and FLAG epitope in the pCMV5 vector. The fusion of the carboxy terminal Ki-Ras4B membrane-association “CAAX” segment (Ser Lys Asp Gly Lys Lys Lys Lys Lys Lys Ser Lys Thr Lys Cys Val Ile Met) (SEQ ID NO:19) to the NORE carboxy terminus to create NORECAAX was accomplished by PCR, using a wild type NORE template, replacing the NORE stop codon with the first amino acid(s) of the CAAX segment. The carboxy terminal pieces of NORE1 (358-413) (i.e., amino acids 348-413 of SEQ ID NO:1) and MST1 (456-487) (i.e., amino acids 456-487 of SEQ ID NO:9) were subcloned inframe downstream of the GST moiety in the pEBG vector (Vavvas et al., J. Biol. Chem., 273: 5439-5442 (1998)) by using the synthetic oligonucleotide (MST1) or PCR (NORE1) approaches. RASSF1A and Ha-RasG12V were cloned into pEBG by shuffling coding sequences from corresponding FLAG-tagged vectors. All other constructs were described in (Vavvas et al., J. Biol. Chem., 273: 5439-5442 (1998)). An amino acid sequence for Raf (i.e., c-Raf1) is shown in SEQ ID NO:20 and a nucleotide sequence encoding Raf is shown in SEQ ID NO:21. All point mutants were made using QUICK CHANGE™ site-directed mutagenesis kit (Stratagene, La Jolla, Calif.). All constructs were verified by DNA sequence analysis.

Tissue Culture and Transfection

COS-7, HEK293 and KB cells were cultivated in Dulbecco's modified Eagle medium supplemented with 10% fetal bovine serum (Sigma, St. Louis, Mo.), 100 units/ml penicillin and 0.1 mg/ml streptomycin in 10% CO₂ atmosphere at 37° C. NIH 3T3 were cultivated as above except 10% Bovine Calf Serum (GIBCO BRL, Gaithersburg, Md.) was used. Cells were replated every third day at the density 10³/cm². For transfection, COS-7 cells were plated at a density of 1.2-2×10⁶ per 10 cm dish and transfected 20-24 hours later with a total of 19 μg of DNA per dish using DEAE-dextran method (Vavvas et al., J. Biol. Chem., 273: 5439-5442 (1998)). HEK293 cells were plated at a density 5-10×10⁶ per 10 cm dish and transfected 20-24 hours later with LIPOFECTAMINE™ transfection reagent (GIBCO BRL, Gaithersburg, Md.) using a total of 8.1 μg of DNA and 40.8 μl LIPOFECTAMINE™ transfection reagent per dish according to manufacturer's instructions. Cells were incubated overnight with the transfection mixture and then fed with the complete growth media or, if required, serum-starved 16-24 hours and harvested before, or at various times after readdition of serum to 10%. Cells were harvested 48 hours after transfection by snap-freezing rinsed plates in liquid nitrogen. KB cells were grown to 80-90% confluence, serum-starved for 24 hours, followed by addition of serum to 10%, and harvest as above. Cell lysis and immunoprecipitation. For the detection of NORE1-MST1 and NORE1-Ras association, frozen cells were scraped into filtered lysis buffer A (30 mM HEPES, pH 7.4, 1% (w/v) Triton X-100, 20 mM β-glycerophosphate, 1 mM orthovanadate, 20 mM NaF, 20 mM KCl, 2 mM EGTA, 3 mM EDTA, 7.5 mM MgCl₂, 14 mM β-mercaptoethanol and a protease inhibitor cocktail (Sigma, St. Louis, Mo.). Lysis buffer B (see below) was used occasionally; no differences were found. Lysates were centrifuged at 17,000×g for 20 minutes. Supernatants were mixed with GSH-agarose (Pharmacia), M2 FLAG-agarose (Sigma) or antibodies and incubated at 4° C. for 2.5-4 hours. When soluble antibodies were used, protein A/G Sepharose (Santa Cruz) was added thereafter for the additional 1.5 hours. Beads were extensively washed in lysis buffer with 25 μM ZnCl₂ and eluted directly into SDS sample buffer. Extracted proteins were separated by SDS-PAGE, transferred onto PVDF membranes, and probed with the antibodies indicated. Bound antibodies were visualized with ECL (Pierce).

The comparison of NORE1 association with MST1, GCK and SOK1, employed lysis buffer B as above with the following changes: supernatants were incubated with M2 FLAG-agarose (Sigma) for 3-4 hours at 4° C. on a rotator. The beads were then washed 6 times with 1 ml of lysis buffer B containing 25 ,μM ZnCl₂ and eluted 3 times, by incubation in lysis buffer B containing 0.1 mg/ml FLAG peptide (Sigma cat #F3290) for 10 min on ice. The eluates were combined and centrifuged for 5 minutes at 5000×g through Spin-X centrifuge tube filters (Coming, cat #8160). To examine Ras-MST1 association, the procedure described above was changed as follows: frozen cells were scraped into lysis buffer A and thawed once; adsorbtion to anti Ras Y13-238 antibodies coupled to agarose was carried out overnight, using 40 μl of settled beads (Santa Cruz); after washing, beads were eluted 3 times each with 40 μl of 0.1 M Glycine-HCl, pH 2.5. The eluates were collected into tubes containing 6 μl of 1.5 M Tris-HCl, pH 8.8 and 6 μl of 10% Triton X-100, centrifuged through SpinX filters (see above) and precipitated with 4 volumes of cold acetone at −20° C. overnight. The precipitated proteins were redissolved in SDS buffer.

MST1 kinase assays. 293 cells on 10 cm plates were transfected with FLAG-tagged MST1 variants and other cDNAs, (total 8.1 μg per plate), using LIPOFECTAMINE™ transfection reagent. All transfections for the purpose of MST kinase assay contained plasmid encoding BVp35CrmA, so as to avoid caspase-mediated cleavage and activation of MST1. Cells were serum-starved for 20-24 hours and serum-stimulated as indicated; rinsed cells were frozen and stored as described above. Frozen cells were scraped into filtered lysis buffer B (30 mM HEPES, pH 7.4, 1% (w/v) Triton X-100, 20 mM β-glycerophosphate, 1 mM orthovanadate, 20 mM KCl, 1 mM EDTA, 1 mM dithiotreitol, 50 μM Calyculin A (GIBCO BRL, Gaithersburg, Md.) and protease inhibitor cocktail (Sigma, St. Louis, Mo.). Lysates were centrifuged at 17 000×g for 20 minutes and supernatants were incubated with anti-FLAG agarose (Sigma) or goat anti-MST1 antibody (Santa Cruz, cat. no. sc-6213) or Zymed anti-MST1 monoclonal (cat. no. 33-3000) as indicated for 3-4 hours at 4° C. on a rotator. The immunoprecipitates were washed 6 times with 1 ml of lysis buffer B containing 25 μM ZnCl₂ and eluted from the beads 3 times, by incubation for 10 min on ice in lysis buffer B containing 0.1 mg/ml FLAG peptide (Sigma cat #F3290) or 18 μg/ml of the peptide used for goat anti MST1 antibody production (SC-6213P), or the peptide used for the monoclonal antibody production. The eluates were combined and centrifuged for 5 minutes at 5000×g through Spin-X centrifuge tube filters (Corning, cat. no. 8160). The flow-through was mixed with an equal volume of glycerol and stored at −20° C. for up to two weeks without apparent loss of the kinase activity, or aliquots were snap-frozen and stored at −80° C. The kinase assay was performed essentially as previously described (Creasy et al., J. Biol. Chem., 271: 21049-21053 (1996)). Briefly, equal amounts of MST1 polypeptide as determined by Western blotting, were incubated in 30 μl of assay buffer containing 40 mM HEPES, pH 7.5, 10 mM MgCl₂, 1 mg/ml MBP (Upstate Biotechnology, cat. no. 13-104), 40 μM ATP, and 2-3 μCi of (γ-32P) ATP for 15 minutes at 30° C. The reaction was stopped by addition of sample buffer to 1× concentration followed by boiling for 2 minutes. An aliquot of the sample was separated by SDS-PAGE on a 12.5% acrylamide gel. The MBP bands were excised after fixation and Coomasie Blue staining and subjected to liquid scintillation counting. The MST1 kinase activity employed in the assays shown was in the linear range as determined by pilot experiments.

Anoptosis Assays

Annexin V labeling in NIH 3T3 cells. The assay was as described in (van Engeland et al., Cytometry, 24: 131-139 (1996)) with some modifications. NIH 3T3 cells were plated at 3-4×10⁵ per well in a 6-well plate the day before transfection. Cells were transfected in duplicate with 6 μg of DNA and 10 μl of LIPOFECTAMINE™ 2000 transfection reagent (GIBCO BRL, Gaithersburg, Md.) in the presence of 10% serum, as recommended by manufacturer, and left for 19-23 hours. In some experiments, Z-Vad-FMK (Calbiochem) was added to 42 μM (final concentration) at the time of transfection. Rat annexinV, purified from liver or recombinant, labeled with Alexa 488 dye was added to the culture media to 1 mg/ml and cells were incubated 5 minutes at 37° C., followed by 25 minutes at room temperature in the dark. Media with floating cells was transferred to 5 ml centrifuge tubes on ice; the plates were washed twice with 1 ml of HBS (20 mM HEPES, pH 7.4, 150 mM NaCl, 2.5 mM CaCl₂) and the washes were added to the tubes with media. Floating cells were pelleted, resuspended in 3 ml of HBS and pelleted again. Adherent cells were scraped into 1 ml HBS, pooled with floating cells from the same dish and resuspended by vortexing. As Jurkat cells are maintained in suspension, the annexin V labeling and washing was carried out as with the “floating” NIH 3T3 cells. Cells were analyzed by Coulter EPICS V XL flow cytometer; the percent of annexinV-positive cells was calculated by the cytometer software. Rat annexinV was purified from frozen rat livers (Pel-Freeze, Ark.) as described (Boustead et al., Biochem. J., 291: 601-608 (1993)) and labeled with Alexa 488 using the Molecular Probes Alexa fluor 488 protein labeling kit according to manufacturer's instructions. In some experiments, the recombinant annexin V used was a kind gift of Dr. M. Schlissel (University of California, Berkeley) that we labeled with Alexa 488.

The Jurkat/T antigen cell line was cultured in complete IMDM medium supplemented with 10% FCS, 2 mM glutamine and gentamycin (50 μg/ml) in a humidified incubator with 5% CO₂ in air at 37° C. The cells were kept at a density of 0.5-1×10⁶/ml. Aliquots containing 3×10⁶ Jurkat cells were electorporated at 250 volts and 960 μF with mixtures of plasmids as indicated in the Figures together with 1 μg of cDNA encoding GFP. Twenty-four hours post transfection, cell survival was analyzed by flow cytometry using a previously optimized program to measure percentage of live cells that are GFP positive present in forward scatter/side scatter gate in each experimental condition (Vermes et al., J. Immunol. Meth., 243: 167-190 (2000)). The vital GFP positive cells were selected on the basis of unchanged scatter signals when compared to untransfected cells and the apoptotic cell population with decreased FS and increased SS was excluded from the analysis. The data was expressed as “percent cells surviving” with vector controls set to 100%.

Apoptosis assays in HEK 293T cells were carried out as described previously (Rabizadeh et al., Cell Death Differ., 6: 1222-1227 (1999); Sperandio et al., Proc. Natl. Acad. Sci. USA, 97: 14376-14381 (2000)) cells were maintained in Dulbecco's Modified Eagle Medium (DMEM) containing 10% fetal bovine serum (FBS). Transient transfections were performed using LIPOFECTAMINE™ transfection reagent (Gibco BRL, Gaithersburg, Md.) according to the manufacturer's instructions. A total of 2.5×10⁵ 293 T cells were seeded in each well of 24-well plates one day prior to transfection. Each well was transfected with a total of 0.5 μg DNA and 3.5 μl LIPOFECTAMINE™ transfection reagent. After one day incubation, the pro-apoptotic agent tamoxifen was added at a final concentration of 20-25 μM to increase the rate of apoptosis. Floating cells were collected at 20-24 hours after the addition of tamoxifen and cell death was assessed using a standard trypan blue exclusion as previously described ((Rabizadeh et al., Cell Death Differ., 6: 1222-1227 (1999); Sperandio et al., Proc. Natl. Acad. Sci. USA, 97: 14376-14381 (2000)).

Effect of NORE1 Expression on Growth of A549 Lung Cancer Cells.

A cDNA insert encoding fall length mouse NORE1 (cloned from a mouse brain cDNA library (Clontech of BD Biosciences, Palo Alto, Calif.) was subeloned into the expression vector pCDNA3.1 (Invitrogen). GENEJUICE™ transfection reagent was purchased from Novagen (Novagen, Inc., Madison, Wis.).

The effect of enhanced expression of NORE1 in a cancer cell line was studied in a standard focus assay. Cells of the A549 cell line (non-small cell lung cancer line harboring an activating K-ras mutation) were grown in six well plate in F12 (Ham's) medium containing 10% fetal calf serum. pCDNA3.1 or pCDNA3.1-NORE1 (0.2 μg/well) plasmid was transfected into 30% confluent A549 cells using the GENEJUICE™ transfection reagent. 48 hours after transfection, the culture medium was changed to F12 (Ham's) containing 10% fetal calf serum plus 2 mg/ml G418. Cells were selected in the presence of G418 for another two weeks (medium refreshed every 4 days). Cells were then washed twice with PBS, fixed with 80% ethanol and stained with 1% crystal violet.

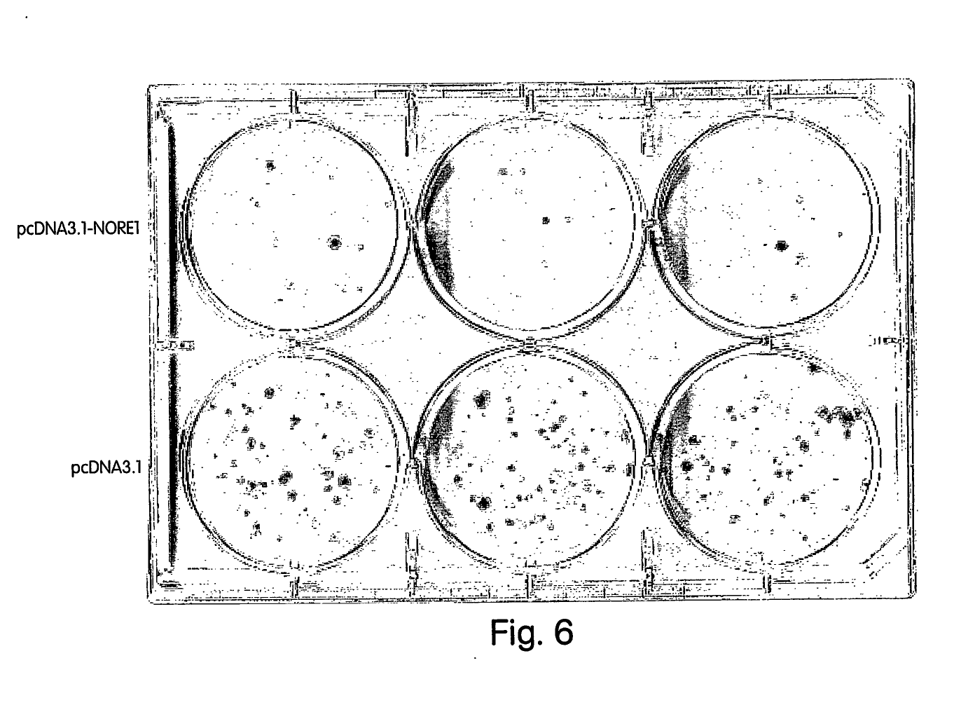

Results are shown in FIG. 6. Since NORE1 expression was found to be very low in A549 cells, it was reasoned that, in view of the above findings, forced expression of NORE1 (i.e., enhanced expression from plasmid pcDNA3.1-NORE1) in those cells might cause cell death or inhibit cell growth. The number of cell colonies that survived G418 selection (representing cells that are actually transfected) was much lower in wells containing pcDNA3.1-NORE1 transfected cells compared to control cells transfected with the pcDNA3.1 vector alone. The results indicated that enhanced expression of NORE1 in A549 cells either caused cell death or inhibited cell growth, i.e., NORE1 functioned as a tumor suppressor. Similar results were obtained when a human melanoma cell line G361, which also exhibits very low expression of NORE1, was used.

All patents, applications, and publications cited in the above text are incorporated herein by reference.

Other variations and embodiments of the invention described herein will now be apparent to those of ordinary skill in the art without departing from the scope and spirit of the invention or the claims below.

1. A method of identifying or evaluating a compound that induces a Ras-mediated pathway of apoptosis in eukaryotic cells comprising:
a) providing eukaryotic cells comprising genetic information for expressing a NORE protein, an MST1 or MST2 protein kinase, a Ras protein, and, optionally, an RASSF1A protein;
b) contacting said eukaryotic cells with a test compound; and
c) assaying said eukaryotic cells contacted with said test compound for an increased level of formation of a complex comprising NORE, MST1 or MST2, and Ras proteins or, optionally, of RASSF1A, MST1 or MST2, and Ras proteins, compared to the level of formation of said complex in cells not contacted with said test compound;
wherein an increase in the level of said complex formation indicates that said test compound induces a Ras-mediated apoptotic pathway in eukaryotic cells.

2. A method of identifying or evaluating a compound that stimulates a Ras-mediated pathway of apoptosis in eukaryotic cells comprising:
a) providing eukaryotic cells comprising genetic information for expressing a NORE protein;
b) contacting said eukaryotic cells with a test compound; and
c) assaying said eukaryotic cells contacted with said test compound for increased expression of NORE protein or increased synthesis of NORE-encoding mRNA compared to cells not contacted with said test compound;
wherein an increase in NORE protein expression or in synthesis of NORE-encoding mRNA indicates that said test compound induces a Ras-mediated pathway of apoptosis in eukaryotic cells.

3. A method of identifying or evaluating a compound that stimulates a Ras-mediated pathway of apoptosis in eukaryotic cells comprising:
a) providing eukaryotic cells comprising genetic information for expressing an RASSF1A protein;
b) contacting said eukaryotic cells with a test compound; and
c) assaying said eukaryotic cells contacted with said test compound for increased expression of RASSFLA protein or increased synthesis of RASSF1A-encoding mRNA compared to cells not contacted with said test compound;
wherein an increase in RASSF1A protein expression or in synthesis of RASSF1A-encoding mRNA indicates that said test compound stimulates a Ras-mediated pathway of apoptosis in eukaryotic cells.

4. A method of identifying or evaluating a compound that stimulates a pathway of apoptosis in eukaryotic cells comprising:
a) providing eukaryotic cells comprising genetic information for expressing an MST1 or MST2 protein kinase;
b) contacting said eukaryotic cells with a test compound;
c) assaying said eukaryotic cells contacted with said test compound for increased expression of MST1 or MST2 protein or increased synthesis of MST1- or MST2-encoding mRNA compared to cells not contacted with said test compound;
wherein an increase in MST1 or MST2 expression or in synthesis of MST1- or MST2-encoding mRNA indicates that said test compound stimulates a pathway of apoptosis in eukaryotic cells.

5. The method according to any one of claims 1-4, further comprising the step:
d) assaying said test compound for the ability to induce apoptosis in a cell of interest in culture.

6. The method according to claim 5, wherein said cell of interest is a cancer cell.

7. The method according to claim 6, wherein said cancer cell is selected from the group consisting of a lung cancer cell, a melanoma cancer cell, a myeloma cancer cell, a leukemia cancer cell, a teratoma cancer cell, a kidney cancer cell, a brain cancer cell, a bone cancer cell, a bladder cancer cell, an epithelial cancer cell, a breast cancer cell, a colon cancer cell, a testicular cancer cell, and a prostate cancer cell.

8. The method according to any one of claims 24, wherein step (c) comprises assaying for increased synthesis of a recited protein-encoding mRNA.

9. The method according to claim 8, wherein said assaying for increased synthesis of the recited protein-encoding mRNA is carried out using a reverse transcriptase polymerase chain reaction (RT PCR).

10. The method according to any one of claims 24, wherein step (c) comprises assaying for increased expression of a recited protein.

11. The method according to any one of claims 14, wherein one or more steps is carried out in a multi-well format that permits multiple compounds to be tested simultaneously.

12. The method according to any one of claims 14, wherein one or more steps is carried out using an apparatus that permits said one or more steps to be carried out automatically or semi-automatically.
